SEQUENCE LISTING (91 sequences)

SEQ ID NO: 1 - protein, 5 aa, Artificial Sequence
  elastin pentapeptide
KGGVG

SEQ ID NO: 2 - protein, 5 aa, Artificial Sequence
  Elastin pentapeptide
VGGVG

SEQ ID NO: 3 - protein, 5 aa, Artificial Sequence
  Elastin pentapeptide
GVGVP

SEQ ID NO: 4 - protein, 5 aa, Artificial Sequence
  Elastin pentapeptide
  MISC_FEATURE (4)..(4): X is V, I or K
VPGXG

SEQ ID NO: 5 - protein, 6 aa, Artificial Sequence
  Elastin hexapeptide
VGVAPG

SEQ ID NO: 6 - protein, 7 aa, Artificial Sequence
  Elastin heptapeptide
LGAGGAG

SEQ ID NO: 7 - protein, 9 aa, Artificial Sequence
  Elastin nonapeptide
LGAGGAGVL

SEQ ID NO: 8 - protein, 7 aa, Artificial Sequence
  Elastin derivative
VGVAPGD

SEQ ID NO: 9 - protein, 7 aa, Artificial Sequence
  Elastin derivative
VGVAPGE

SEQ ID NO: 10 - DNA, 1251 bp, Artificial Sequence
  3f-tv-Gtclp-tv-ha
atggcagattataaagacgatgatgataaagattacaaagacgatgacgacaaagactataaagatgatgacgataaagaaaacctttattttcaaggtagtcgtcgtcgtcaaggtgctgctgccgcagccgctggtgcagcaggattaatcttacttgtgttactttcattagtatctttacgtgctcatgaacgcaaaccaacatctttagttgaaggttcacgcactacaatgatgccaacatggaaagtaactggtacattaaatccagttgaagaaacagagaaagaagtagaagaagaaccatcttcacctccaccaccagcagtacaagctacaccagatgtacctcaagagcaagaagccgaaccaagtcagtcagaatgggttccaccacctaaaccttgggaaccattagctgaaaaaaatattggtcgtttagaacgtactaatcgtgaagtagttcgtggtgatatggaaaatgcaaacatgataaaaaaattacgtgctgctatgcgtttattaaagaaacaatttgcagctaaaattgtgaatttagaacaagcttatgatcgtcgtttagctcgtgaaagtcgtattttacaccaacgtattaatacagtagctatgcaacctggtccaacaggcatgtcaggtcctcaaggtttaccaggtcctactggtccagcaggccaaaatggttctccaggctcaccaggagcaatgggtccacaaggaccaatgggtccaagaggttatcgtggtttacgtggtccacctggtgatcaaggtcgtcctggtgatacaggtagacctggtgctcctggtattccaggtcaaattggtcaacgtggacctattggtccaatgggtgttcaaggccctgaaggtccacgtggtaatcctggattaccaggtccaccaggtgcaccaggtgttccaggtattggtattcaaggaccagcaggtcctcctggtccaccaggacgctttcctgctacagaacattgtagctatgttactggtgaatgtgtaattaacggcaatgacattttccatgctatgcgtgaaactggcgatgtaagttgtccaagaaactactatgttaaaggtgttgattacatcaaatgcaataatggtgctgaataccacaatactttacagttacgtttaacatgttgtttattaggtttaaacgagaatctttatttccaaggttatccttatgatgttccagattatgcttaa

SEQ ID NO: 11 - DNA, 18 bp, Artificial Sequence
  nucleic sequence encoding the elastin peptide VGVAPG
gtaggtgtagctcctggt

SEQ ID NO: 12 - DNA, 18 bp, Artificial Sequence
  nucleic sequence encoding the elastin peptide VGVAPG
gttggtgttgctcctgga

SEQ ID NO: 13 - DNA, 18 bp, Artificial Sequence
  nucleic sequence encoding the elastin peptide VGVAPG
gtaggtgttgctccaggt

SEQ ID NO: 14 - DNA, 18 bp, Artificial Sequence
  nucleic sequence encoding the elastin peptide VGVAPG
gtgggtgtagctcctggt

SEQ ID NO: 15 - DNA, 21 bp, Artificial Sequence
  nucleic sequence encoding the elastin peptide derivative VGVAPGE
gtaggtgtagctcctggtgaa

SEQ ID NO: 16 - DNA, 21 bp, Artificial Sequence
  nucleic seuqence encoding the elastin peptide derivative VGVAPGE
gttggtgttgctcctggagaa

SEQ ID NO: 17 - DNA, 21 bp, Artificial Sequence
  nucleic sequence encoding the elastin peptide derivative VGVAPGE
gtaggtgttgctccaggtgaa

SEQ ID NO: 18 - DNA, 21 bp, Artificial Sequence
  nucleic sequence encoding the elastin peptide derivative VGVAPGE
gtgggtgtagctcctggtgaa

SEQ ID NO: 19 - DNA, 852 bp, Artificial Sequence
  ha-sp-3f-Gtccld-3ha
atggcatatccttatgatgtaccagattatgccaataataacgacttatttcaagcatcacgtcgtcgtttcttagctcaattaggaggtttaacagttgctggtatgttaggtccatctttacttactccacgtagagctacagcagctcaagctgattacaaagatgatgacgataaagactataaagatgatgatgacaaagattacaaagacgatgatgataaaggatccggtccatgttgtggaccacctggtcctccaggtccaccaggaccacctggaccaacaggtatgagtggaccacaaggtttaccaggtcctactggtcctgctggtcaaaacggttcaccaggttctccaggtgcaatgggtccacaaggtcctatgggaccacgtggttatcgtggtttacgtggtccaccaggtgatcaaggtcgtcctggtgatacaggtcgtccaggtgctccaggtattccaggtcagattggtcaacgtggtccaattggtcctatgggtgttcaaggaccagaaggtccaagaggtaatccaggccttccaggaccaccaggtgcaccaggtgtacctggtattggcattcaaggtcctgctggcccacctggtccacctggtcgttttggtcctcctggccctccaggcccaccaggtcctcctggtccatgttgcggctatatcccagaagctccacgtgatggtcaagcttatgttcgcaaagatggtgaatgggtgttattatcaacattcttatacccttatgacgtaccagattacgcatatccttatgatgttccagattatgcctatccatacgatgtacctgactatgcttaa

SEQ ID NO: 20 - DNA, 771 bp, Artificial Sequence
  ha-sp-3f-Gtccld
atggcatatccttatgatgtaccagattatgccaataataacgacttatttcaagcatcacgtcgtcgtttcttagctcaattaggaggtttaacagttgctggtatgttaggtccatctttacttactccacgtagagctacagcagctcaagctgattacaaagatgatgacgataaagactataaagatgatgatgacaaagattacaaagacgatgatgataaaggatccggtccatgttgtggaccacctggtcctccaggtccaccaggaccacctggaccaacaggtatgagtggaccacaaggtttaccaggtcctactggtcctgctggtcaaaacggttcaccaggttctccaggtgcaatgggtccacaaggtcctatgggaccacgtggttatcgtggtttacgtggtccaccaggtgatcaaggtcgtcctggtgatacaggtcgtccaggtgctccaggtattccaggtcagattggtcaacgtggtccaattggtcctatgggtgttcaaggaccagaaggtccaagaggtaatccaggccttccaggaccaccaggtgcaccaggtgtacctggtattggcattcaaggtcctgctggcccacctggtccacctggtcgttttggtcctcctggccctccaggcccaccaggtcctcctggtccatgttgcggctatatcccagaagctccacgtgatggtcaagcttatgttcgcaaagatggtgaatgggtgttattatcaacattcttataa

SEQ ID NO: 21 - DNA, 594 bp, Artificial Sequence
  ha-sp-3f-Gtcld
atggcatatccttatgatgtaccagattatgccaataataacgacttatttcaagcatcacgtcgtcgtttcttagctcaattaggaggtttaacagttgctggtatgttaggtccatctttacttactccacgtagagctacagcagctcaagctgattacaaagatgatgacgataaagactataaagatgatgatgacaaagattacaaagacgatgatgataaaggatccggaccaacaggtatgagtggaccacaaggtttaccaggtcctactggtcctgctggtcaaaacggttcaccaggttctccaggtgcaatgggtccacaaggtcctatgggaccacgtggttatcgtggtttacgtggtccaccaggtgatcaaggtcgtcctggtgatacaggtcgtccaggtgctccaggtattccaggtcagattggtcaacgtggtccaattggtcctatgggtgttcaaggaccagaaggtccaagaggtaatccaggccttccaggaccaccaggtgcaccaggtgtacctggtattggcattcaaggtcctgctggcccacctggtccacctggtcgtttttaa

SEQ ID NO: 22 - DNA, 72 bp, Artificial Sequence
  elp4
gtaggtgtagctcctggtgttggtgttgctcctggagtaggtgttgctccaggtgtgggtgtagctcctggt

SEQ ID NO: 23 - DNA, 84 bp, Artificial Sequence
  elpe4
gtaggtgtagctcctggtgaagttggtgttgctcctggagaagtaggtgttgctccaggtgaagtgggtgtagctcctggtgaa

SEQ ID NO: 24 - protein, 8 aa, Artificial Sequence
  Flag Tag
DYKDDDDK

SEQ ID NO: 25 - protein, 24 aa, Artificial Sequence
  3X Flag Tag
DYKDDDDKDYKDDDDKDYKDDDDK

SEQ ID NO: 26 - protein, 9 aa, Artificial Sequence
  HA Tag
YPYDVPDYA

SEQ ID NO: 27 - protein, 27 aa, Artificial Sequence
  3xHA Tag
YPYDVPDYAYPYDVPDYAYPYDVPDYA

SEQ ID NO: 28 - protein, 6 aa, Artificial Sequence
  His Tag
HHHHHH

SEQ ID NO: 29 - DNA, 424 bp, Artificial Sequence
  PpsbD-5′UTR
atgaaattaaatggatatttggtacatttaattccacaaaaatgtccaatacttaaaatacaaaattaaaagtattagttgtaaacttgactaacattttaaattttaaattttttcctaattatatattttacttgcaaaatttataaaaattttatgcatttttatatcataataataaaacctttattcatggtttataatataataattgtgatgactatgcacaaagcagttctagtcccatatatataactatatataacccgtttaaagatttatttaaaaatatgtgtgtaaaaaatgcttatttttaattttattttatataagttataatattaaatacacaatgattaaaattaaataataataaatttaacgtaacgatgagttgtttttttattttggagatacacgcacc

SEQ ID NO: 30 - DNA, 1025 bp, Artificial Sequence
  Prrn-5′UTRatpA
tttttaattaagtaggaactcggtatatgctcttttggggtcttattagctagtattagttaactaacaaaagatcaatattttagtttgttttatatattttattacttaagtagtaaggatttgcatttagcaatcttaaatacttaagtaataatctataaataaaatatattttcgctttaaaacttataaaaattatttgctcgttataagcctaaaaaaacgtaggatctctacgagatattacattgtttttttctttaattggctttaatattactttgtatatataaaccaaagtacttgttaatagttattaaattatattaactatacagtacaaagaaattttttgctaaaaaaagtatgttaacattaaaaatttttgtttatacagactagtcagactcggggggcaggcaacaaatttatttattgtcccgtaaggggaaggggaaaacaattattattttactgcggagcagcttgttattgaaattttattaaaaaaaaaataaaaatttgacaaaaaaaaataaaaaagttaaattaaaaacactgggaatgttctacatcataaaaatcaaaagggtttaaaatcccgacaaaatttaaactttaaagagtggcgcctaccttttttttaatttgcatgattttaatgcttatgctatcttttttatttagtccataaaacctttaaaggaccttttcttatgggatatttatattttcctaacaaagcaatcggcgtcataaactttagttgcttacgacgcctgtggacgtcccccccttccccttacgggcaagtaaacttagggattttaatgcaataaataaatttgtcctcttcgggcaaatgaattttagtatttaaatatgacaagggtgaaccattacttttgttaacaagtgatcttaccactcactatttttgttgaattttaaacttatttaaaattctcgagaaagattttaaaaataaacttttttaatcttttatttattttttctttttt

SEQ ID NO: 31 - DNA, 400 bp, Artificial Sequence
  3′UTRatpA
tttttaattaagtaggaactcggtatatgctcttttggggtcttattagctagtattagttaactaacaaaagatcaatattttagtttgttttatatattttattacttaagtagtaaggatttgcatttagcaatcttaaatacttaagtaataatctataaataaaatatattttcgctttaaaacttataaaaattatttgctcgttataagcctaaaaaaacgtaggatctctacgagatattacattgtttttttctttaattggctttaatattactttgtatatataaaccaaagtacttgttaatagttattaaattatattaactatacagtacaaagaaattttttgctaaaaaaagtatgttaacattaaaaatttttgtttatacag

SEQ ID NO: 32 - DNA, 435 bp, Artificial Sequence
  3′UTRrbcL
aagcttgtactcaagctcgtaacgaaggtcgtgaccttgctcgtgaaggtggcgacgtaattcgttcagcttgtaaatggtctccagaacttgctgctgcatgtgaagtttggaaagaaattaaattcgaatttgatactattgacaaactttaatttttatttttcatgatgtttatgtgaatagcataaacatcgtttttattttttatggtgtttaggttaaatacctaaacatcattttacatttttaaaattaagttctaaagttatcttttgtttaaatttgcctgtgctttataaattacgatgtgccagaaaaataaaatcttagctttttattatagaatttatctttatgtattatattttataagtaataaaagaaatagtaacatactaaagcggatgtaactcaatcggtagagtgcgatcc

SEQ ID NO: 33 - protein, 41 aa, Artificial Sequence
  SP
NNNDLFQASRRRFLAQLGGLTVAGMLGPSLLTPRRATAAQA

SEQ ID NO: 34 - DNA, 123 bp, Artificial Sequence
  nucleic sequence encoding SP
aataataacgacttatttcaagcatcacgtcgtcgtttcttagctcaattaggaggtttaacagttgctggtatgttaggtccatctttacttactccacgtagagctacagcagctcaagct

SEQ ID NO: 35 - protein, 58 aa, Bos taurus [sequence omitted]

SEQ ID NO: 36 - DNA, 174 bp, Bos taurus
cggcctgacttctgcctagagcctccatatacgggtccctgcaaggccagaattatcagatacttctacaacgccaaggctgggctctgccagacctttgtatatggcggctgcagagctaaaagaaacaatttcaagagcgcagaggactgcatgaggacctgtggtggtgct

SEQ ID NO: 37 - protein, 67 aa, Artificial Sequence
  HA-APRO
YPYDVPDYARPDFCLEPPYTGPCKARIIRYFYNAKAGLCQTFVYGGCRAKRNNFKSAEDCMRTCGGA

SEQ ID NO: 38 - protein, 5 aa, Artificial Sequence
  EK
DDDDK

SEQ ID NO: 39 - protein, 85 aa, Artificial Sequence
  3F-APRO
AQADYKDDDDKDYKDDDDKDYKDDDDKRPDFCLEPPYTGPCKARIIRYFYNAKAGLCQTFVYGGCRAKRNNFKSAEDCMRTCGGA

SEQ ID NO: 40 - DNA, 234 bp, Artificial Sequence
  lgm-tv-elpe4
cagctgaagattgtatgcgtacttgtggtggtgctagatctggtggaggcggttcaagtggtggaggaggtggcggatcttcaagatctgaaaatttatattttcaaggtgtaggtgtagctcctggtgaagttggtgttgctcctggagaagtaggtgttgctccaggtgaagtgggtgtagctcctggtgaataagtttaaaccaggtgacctgcagagctagctactgcag

SEQ ID NO: 41 - protein, 140 aa, Artificial Sequence
  HA-SP-3F-FX-APRO
MAYPYDVPDYANNNDLFQASRRRFLAQLGGLTVAGMLGPSLLTPRRATAAQADYKDDDDKDYKDDDDKDYKDDDDKGSIEGRRPDFCLEPPYTGPCKARIIRYFYNAKAGLCQTFVYGGCRAKRNNFKSAEDCMRTCGGA

SEQ ID NO: 42 - DNA, 423 bp, Artificial Sequence
  nucleic sequence coding for HA-SP-3F-FX-APRO
atggcatatccttatgatgtaccagattatgccaataataacgacttatttcaagcatcacgtcgtcgtttcttagctcaattaggaggtttaacagttgctggtatgttaggtccatctttacttactccacgtagagctacagcagctcaagctgattacaaagatgatgacgataaagactataaagatgatgatgacaaagattacaaagacgatgatgataaaggatccattgaaggtcgtagaccagacttttgcttagaaccaccatatacaggtccatgtaaagctcgtatcattcgctatttctacaatgcaaaagcaggattatgtcaaacatttgtgtatggaggttgtcgtgctaaacgtaacaactttaaatcagctgaagattgtatgcgtacttgtggtggtgcttaa

SEQ ID NO: 43 - protein, 134 aa, Artificial Sequence
  HA-SP-3F-APRO
MAYPYDVPDYANNNDLFQASRRRFLAQLGGLTVAGMLGPSLLTPRRATAAQADYKDDDDKDYKDDDDKDYKDDDDKRPDFCLEPPYTGPCKARIIRYFYNAKAGLCQTFVYGGCRAKRNNFKSAEDCMRTCGGA

SEQ ID NO: 44 - DNA, 349 bp, Artificial Sequence
  nucleic sequence encoding HA-SP-3F-APRO
atcacgtcgtcgtttcttagctcaattaggaggtttaacagttgctggtatgttaggtccatctttacttactccacgtagagctacagcagctcaagctgattacaaagatgatgacgataaagactataaagatgatgatgacaaagattacaaagacgatgatgataaaagaccagacttttgcttagaaccaccatatacaggtccatgtaaagctcgtatcattcgctatttctacaatgcaaaagcaggattatgtcaaacatttgtgtatggaggttgtcgtgctaaacgtaacaactttaaatcagctgaagattgtatgcgtacttgtggtggtgcttaa

SEQ ID NO: 45 - protein, 108 aa, Artificial Sequence
  HA-SP-APRO
YPYDVPDYANNNDLFQASRRRFLAQLGGLTVAGMLGPSLLTPRRATAAQARPDFCLEPPYTGPCKARIIRYFYNAKAGLCQTFVYGGCRAKRNNFKSAEDCMRTCGGA

SEQ ID NO: 46 - DNA, 324 bp, Artificial Sequence
  nucleic sequence encoding for HA-SP-APRO
tatccttatgatgtaccagattatgccaataataacgacttatttcaagcatcacgtcgtcgtttcttagctcaattaggaggtttaacagttgctggtatgttaggtccatctttacttactccacgtagagctacagcagctcaagctagaccagacttttgcttagaaccaccatatacaggtccatgtaaagctcgtatcattcgctatttctacaatgcaaaagcaggattatgtcaaacatttgtgtatggaggttgtcgtgctaaacgtaacaactttaaatcagctgaagattgtatgcgtacttgtggtggtgct

SEQ ID NO: 47 - protein, 91 aa, Artificial Sequence
  3F-FX-APRO
AQADYKDDDDKDYKDDDDKDYKDDDDKGSIEGRRPDFCLEPPYTGPCKARIIRYFYNAKAGLCQTFVYGGCRAKRNNFKSAEDCMRTCGGA

SEQ ID NO: 48 - DNA, 231 bp, Artificial Sequence
  lgm-ek-elpe4
cagctgaagattgtatgcgtacttgtggtggtgctagatctggtggaggcggttcaagtggtggaggaggtggcggatcttcaagatctgacgatgacgacaagggtgtaggtgtagctcctggtgaagttggtgttgctcctggagaagtaggtgttgctccaggtgaagtgggtgtagctcctggtgaataagtttaaaccaggtgacctgcagagctagctactgcag

SEQ ID NO: 49 - protein, 14 aa, Artificial Sequence
  LG
RSGGGGSGGGGSGS

SEQ ID NO: 50 - protein, 18 aa, Artificial Sequence
  LGM
RSGGGGSSGGGGGGSSRS

SEQ ID NO: 51 - protein, 365 aa, Artificial Sequence
  CCMP2712
MSRRRQGAAAAAAGAAGLILLVLLSLVSLRAHERKPTSLVEGSRTTMMPTWKVTGTLNPVEETEKEVEEEPSSPPPPAVQATPDVPQEQEAEPSQSEWVPPPKPWEPLAEKNIGRLERTNREVVRGDMENANMIKKLRAAMRLLKKQFAAKIVNLEQAYDRRLARESRILHQRINTVAMQPGPTGMSGPQGLPGPTGPAGQNGSPGSPGAMGPQGPMGPRGYRGLRGPPGDQGRPGDTGRPGAPGIPGQIGQRGPIGPMGVQGPEGPRGNPGLPGPPGAPGVPGIGIQGPAGPPGPPGRFPATEHCSYVTGECVINGNDIFHAMRETGDVSCPRNYYVKGVDYIKCNNGAEYHNTLQLRLTCCLL

SEQ ID NO: 52 - protein, 7 aa, Artificial Sequence
  TEV protease
ENLYFQG

SEQ ID NO: 53 - protein, 29 aa, Artificial Sequence
  polypeptide of example 3
GVGVAPGEVGVAPGEVGVAPGEVGVAPGE

SEQ ID NO: 54 - DNA, 34 bp, Artificial Sequence
  O3′SUTRpsbD
cgatgagttgtttttttattttggagatacacgc

SEQ ID NO: 55 - protein, 416 aa, Artificial Sequence
  3F-TV-GtCLP-TV-HA
MADYKDDDDKDYKDDDDKDYKDDDDKENLYFQGSRRRQGAAAAAAGAAGLILLVLLSLVSLRAHERKPTSLVEGSRTTMMPTWKVTGTLNPVEETEKEVEEEPSSPPPPAVQATPDVPQEQEAEPSQSEWVPPPKPWEPLAEKNIGRLERTNREVVRGDMENANMIKKLRAAMRLLKKQFAAKIVNLEQAYDRRLARESRILHQRINTVAMQPGPTGMSGPQGLPGPTGPAGQNGSPGSPGAMGPQGPMGPRGYRGLRGPPGDQGRPGDTGRPGAPGIPGQIGQRGPIGPMGVQGPEGPRGNPGLPGPPGAPGVPGIGIQGPAGPPGPPGRFPATEHCSYVTGECVINGNDIFHAMRETGDVSCPRNYYVKGVDYIKCNNGAEYHNTLQLRLTCCLLGLNENLYFQGYPYDVPDYA

SEQ ID NO: 56 - DNA, 62 bp, Artificial Sequence
  O5′SCL70
ctgcagtagctagctctgcaggtcacctgttaagcataatctggaacatcataaggataacc

SEQ ID NO: 57 - DNA, 62 bp, Artificial Sequence
  O3′SCL70
cgatgagttgtttttttattttggagatacacgcaccatggcagattataaagacgatgatg

SEQ ID NO: 58 - DNA, 1317 bp, Artificial Sequence
  FPCR-SCL70
ctgcagtagctagctctgcaggtcacctgttaagcataatctggaacatcataaggataaccttggaaataaagattctcgtttaaacctaataaacaacatgttaaacgtaactgtaaagtattgtggtattcagcaccattattgcatttgatgtaatcaacacctttaacatagtagtttcttggacaacttacatcgccagtttcacgcatagcatggaaaatgtcattgccgttaattacacattcaccagtaacatagctacaatgttctgtagcaggaaagcgtcctggtggaccaggaggacctgctggtccttgaataccaatacctggaacacctggtgcacctggtggacctggtaatccaggattaccacgtggaccttcagggccttgaacacccattggaccaataggtccacgttgaccaatttgacctggaataccaggagcaccaggtctacctgtatcaccaggacgaccttgatcaccaggtggaccacgtaaaccacgataacctcttggacccattggtccttgtggacccattgctcctggtgagcctggagaaccattttggcctgctggaccagtaggacctggtaaaccttgaggacctgacatgcctgttggaccaggttgcatagctactgtattaatacgttggtgtaaaatacgactttcacgagctaaacgacgatcataagcttgttctaaattcacaattttagctgcaaattgtttctttaataaacgcatagcagcacgtaatttttttatcatgtttgcattttccatatcaccacgaactacttcacgattagtacgttctaaacgaccaatatttttttcagctaatggttcccaaggtttaggtggtggaacccattctgactgacttggttcggcttcttgctcttgaggtacatctggtgtagcttgtactgctggtggtggaggtgaagatggttcttcttctacttctttctctgtttcttcaactggatttaatgtaccagttactttccatgttggcatcattgtagtgcgtgaaccttcaactaaagatgttggtttgcgttcatgagcacgtaaagatactaatgaaagtaacacaagtaagattaatcctgctgcaccagcggctgcggcagcagcaccttgacgacgacgactaccttgaaaataaaggttttctttatcgtcatcatctttatagtctttgtcgtcatcgtctttgtaatctttatcatcatcgtctttataatctgccatggtgcgtgtatctccaaaataaaaaaacaactcatcg

SEQ ID NO: 59 - protein, 119 aa, Artificial Sequence
  collagen-like domain from G. theta CCMP2712
GPTGMSGPQGLPGPTGPAGQNGSPGSPGAMGPQGPMGPRGYRGLRGPPGDQGRPGDTGRPGAPGIPGQIGQRGPIGPMGVQGPEGPRGNPGLPGPPGAPGVPGIGIQGPAGPPGPPGRF

SEQ ID NO: 60 - protein, 16 aa, Artificial Sequence
  Cys Knot and CR4 Repeat sequences
GPCCGPPGPPGPPGPP

SEQ ID NO: 61 - protein, 27 aa, Artificial Sequence
  Foldon fibritin of the T4 phage
GYIPEAPRDGQAYVRKDGEWVLLSTFL

SEQ ID NO: 62 - protein, 180 aa, Artificial Sequence
  GtCCLD
GSGPCCGPPGPPGPPGPPGPTGMSGPQGLPGPTGPAGQNGSPGSPGAMGPQGPMGPRGYRGLRGPPGDQGRPGDTGRPGAPGIPGQIGQRGPIGPMGVQGPEGPRGNPGLPGPPGAPGVPGIGIQGPAGPPGPPGRFGPPGPPGPPGPPGPCCGYIPEAPRDGQAYVRKDGEWVLLSTFL

SEQ ID NO: 63 - protein, 27 aa, Artificial Sequence
  3HA
YPYDVPDYAYPYDVPDYAYPYDVPDYA

SEQ ID NO: 64 - protein, 207 aa, Artificial Sequence
  GtCCLD-3HA
GSGPCCGPPGPPGPPGPPGPTGMSGPQGLPGPTGPAGQNGSPGSPGAMGPQGPMGPRGYRGLRGPPGDQGRPGDTGRPGAPGIPGQIGQRGPIGPMGVQGPEGPRGNPGLPGPPGAPGVPGIGIQGPAGPPGPPGRFGPPGPPGPPGPPGPCCGYIPEAPRDGQAYVRKDGEWVLLSTFLYPYDVPDYAYPYDVPDYAYPYDVPDYA

SEQ ID NO: 65 - DNA, 617 bp, Artificial Sequence
  PCR fragment Gtccld-2
gatgatgatgacaaagattacaaagacgatgatgataaaggatccggtccatgttgtggaccacctggtcctccaggtccaccaggaccacctggaccaacaggtatgagtggaccacaaggtttaccaggtcctactggtcctgctggtcaaaacggttcaccaggttctccaggtgcaatgggtccacaaggtcctatgggaccacgtggttatcgtggtttacgtggtccaccaggtgatcaaggtcgtcctggtgatacaggtcgtccaggtgctccaggtattccaggtcagattggtcaacgtggtccaattggtcctatgggtgttcaaggaccagaaggtccaagaggtaatccaggccttccaggaccaccaggtgcaccaggtgtacctggtattggcattcaaggtcctgctggcccacctggtccacctggtcgttttggtcctcctggccctccaggcccaccaggtcctcctggtccatgttgcggctatatcccagaagctccacgtgatggtcaagcttatgttcgcaaagatggtgaatgggtgttattatcaacattcttataagtttaaacgtcgacctgcagagctagctactgcag

SEQ ID NO: 66 - DNA, 440 bp, Artificial Sequence
  PCR fragment Gtcld
gatgatgatgacaaagattacaaagacgatgatgataaaggatccggaccaacaggtatgagtggaccacaaggtttaccaggtcctactggtcctgctggtcaaaacggttcaccaggttctccaggtgcaatgggtccacaaggtcctatgggaccacgtggttatcgtggtttacgtggtccaccaggtgatcaaggtcgtcctggtgatacaggtcgtccaggtgctccaggtattccaggtcagattggtcaacgtggtccaattggtcctatgggtgttcaaggaccagaaggtccaagaggtaatccaggccttccaggaccaccaggtgcaccaggtgtacctggtattggcattcaaggtcctgctggcccacctggtccacctggtcgtttttaagtttaaacgtcgacctgcagagctagctactgcag

SEQ ID NO: 67 - DNA, 35 bp, Artificial Sequence
  O5′ASTatpA2
cctacttaattaaaaactgcagtagctagctctgc

SEQ ID NO: 68 - DNA, 34 bp, Artificial Sequence; identical to SEQ ID NO: 54
  O3′SUTRpsbD

SEQ ID NO: 69 - DNA, 62 bp, Artificial Sequence; identical to SEQ ID NO: 56
  O5′SCL70

SEQ ID NO: 70 - DNA, 33 bp, Artificial Sequence
  O5′PpsbDCla2
catcgatgatgaaattaaatggatatttggtac

SEQ ID NO: 71 - protein, 4 aa, Artificial Sequence
  FX
IEGR

SEQ ID NO: 72 - DNA, 116 bp, Artificial Sequence
  O5′Gibs-ELP4
ctgcagtagctagctctgcaggtcgacgtttaaacttaagcgtaatctggaacatcatatggataaccaggagctacacccacacctggagcaacacctactccaggagcaacacc

SEQ ID NO: 73 - DNA, 110 bp, Artificial Sequence
  O3′Gibs-ELP4
gctgaagattgtatgcgtacttgtggtggtgctgattacaaagacgatgatgacaaagtaggtgtagctcctggtgttggtgttgctcctggagtaggtgttgctccagg

SEQ ID NO: 74 - DNA, 62 bp, Artificial Sequence
  O5′Gibs01BE
ctgcagtagctagctctgcaggtcgacgtttaaacttaaccaggagctacacccacacctgg

SEQ ID NO: 75 - DNA, 70 bp, Artificial Sequence
  O3′Gibs01BE
gatgacaaagattacaaagacgatgatgataaaggatccattgaaggtcgtagaccagacttttgcttag

SEQ ID NO: 76 - DNA, 359 bp, Artificial Sequence
  FPCR-AP-FELP4
gatgacaaagattacaaagacgatgatgataaaggatccattgaaggtcgtagaccagacttttgcttagaaccaccatatacaggtccatgtaaagctcgtatcattcgctatttctacaatgcaaaagcaggattatgtcaaacatttgtgtatggaggttgtcgtgctaaacgtaacaactttaaatcagctgaagattgtatgcgtacttgtggtggtgctgattacaaagacgatgatgacaaagtaggtgtagctcctggtgttggtgttgctcctggagtaggtgttgctccaggtgtgggtgtagctcctggttaagtttaaacgtcgacctgcagagctagctactgcag

SEQ ID NO: 77 - DNA, 207 bp, Artificial Sequence
  FPCR-FELP4-HA
agatgatgatgacaaagattacaaagacgatgatgataaaggatccgattacaaagacgatgatgacaaagtaggtgtagctcctggtgttggtgttgctcctggagtaggtgttgctccaggtgtgggtgtagctcctggttatccatatgatgttccagattacgcttaagtttaaacgtcgacctgcagagctagctactgcag

SEQ ID NO: 78 - DNA, 62 bp, Artificial Sequence
  O5′Gibs02BE
ctgcagtagctagctctgcaggtcgacgtttaaacttaagcgtaatctggaacatcatatgg

SEQ ID NO: 79 - DNA, 75 bp, Artificial Sequence
  O3′Gibs02BE
agatgatgatgacaaagattacaaagacgatgatgataaaggatccgattacaaagacgatgatgacaaagtagg

SEQ ID NO: 80 - protein, 28 aa, Artificial Sequence
  ELPE4
VGVAPGEVGVAPGEVGVAPGEVGVAPGE

SEQ ID NO: 81 - protein, 24 aa, Artificial Sequence
  ELP4
VGVAPGVGVAPGVGVAPGVGVAPG

SEQ ID NO: 82 - DNA, 1282 bp, Artificial Sequence
  expression cassette of example 3
atgaaattaaatggatatttggtacatttaattccacaaaaatgtccaatacttaaaatacaaaattaaaagtattagttgtaaacttgactaacattttaaattttaaattttttcctaattatatattttacttgcaaaatttataaaaattttatgcatttttatatcataataataaaacctttattcatggtttataatataataattgtgatgactatgcacaaagcagttctagtcccatatatataactatatataacccgtttaaagatttatttaaaaatatgtgtgtaaaaaatgcttatttttaattttattttatataagttataatattaaatacacaatgattaaaattaaataataataaatttaacgtaacgatgagttgtttttttattttggagatacacgcaccatggcatatccttatgatgtaccagattatgccaataataacgacttatttcaagcatcacgtcgtcgtttcttagctcaattaggaggtttaacagttgctggtatgttaggtccatctttacttactccacgtagagctacagcagctcaagctgattacaaagatgatgacgataaagactataaagatgatgatgacaaagattacaaagacgatgatgataaaggatccattgaaggtcgtagaccagacttttgcttagaaccaccatatacaggtccatgtaaagctcgtatcattcgctatttctacaatgcaaaagcaggattatgtcaaacatttgtgtatggaggttgtcgtgctaaacgtaacaactttaaatcagctgaagattgtatgcgtacttgtggtggtgcttaagtttaaacgtcgacctgcagagctagctactgcagtttttaattaagtaggaactcggtatatgctcttttggggtcttattagctagtattagttaactaacaaaagatcaatattttagtttgttttatatattttattacttaagtagtaaggatttgcatttagcaatcttaaatacttaagtaataatctataaataaaatatattttcgctttaaaacttataaaaattatttgctcgttataagcctaaaaaaacgtaggatctctacgagatattacattgtttttttctttaattggctttaatattactttgtatatataaaccaaagtacttgttaatagttattaaattatattaactatacagtacaaagaaattttttgctaaaaaaagtatgttaacattaaaaatttttgtttatacag

SEQ ID NO: 83 - protein, 283 aa, Artificial Sequence
  HA-SP-3F-GtCCLD-3HA
MAYPYDVPDYANNNDLFQASRRRFLAQLGGLTVAGMLGPSLLTPRRATAAQADYKDDDDKDYKDDDDKDYKDDDDKGSGPCCGPPGPPGPPGPPGPTGMSGPQGLPGPTGPAGQNGSPGSPGAMGPQGPMGPRGYRGLRGPPGDQGRPGDTGRPGAPGIPGQIGQRGPIGPMGVQGPEGPRGNPGLPGPPGAPGVPGIGIQGPAGPPGPPGRFGPPGPPGPPGPPGPCCGYIPEAPRDGQAYVRKDGEWVLLSTFLYPYDVPDYAYPYDVPDYAYPYDVPDYA

SEQ ID NO: 84 - DNA, 652 bp, Artificial Sequence
  synthetic codon optimized gene Gtccld-3ha
gtccatggctggatccggtccatgttgtggaccacctggtcctccaggtccaccaggaccacctggaccaacaggtatgagtggaccacaaggtttaccaggtcctactggtcctgctggtcaaaacggttcaccaggttctccaggtgcaatgggtccacaaggtcctatgggaccacgtggttatcgtggtttacgtggtccaccaggtgatcaaggtcgtcctggtgatacaggtcgtccaggtgctccaggtattccaggtcagattggtcaacgtggtccaattggtcctatgggtgttcaaggaccagaaggtccaagaggtaatccaggccttccaggaccaccaggtgcaccaggtgtacctggtattggcattcaaggtcctgctggcccacctggtccacctggtcgttttggtcctcctggccctccaggcccaccaggtcctcctggtccatgttgcggctatatcccagaagctccacgtgatggtcaagcttatgttcgcaaagatggtgaatgggtgttattatcaacattcttatacccttatgacgtaccagattacgcatatccttatgatgttccagattatgcctatccatacgatgtacctgactatgcttaagtttaaacgtcgacctgc

SEQ ID NO: 85 - protein, 256 aa, Artificial Sequence
  HA-SP-3F-GtCCLD
MAYPYDVPDYANNNDLFQASRRRFLAQLGGLTVAGMLGPSLLTPRRATAAQADYKDDDDKDYKDDDDKDYKDDDDKGSGPCCGPPGPPGPPGPPGPTGMSGPQGLPGPTGPAGQNGSPGSPGAMGPQGPMGPRGYRGLRGPPGDQGRPGDTGRPGAPGIPGQIGQRGPIGPMGVQGPEGPRGNPGLPGPPGAPGVPGIGIQGPAGPPGPPGRFGPPGPPGPPGPPGPCCGYIPEAPRDGQAYVRKDGEWVLLSTFL

SEQ ID NO: 86 - protein, 197 aa, Artificial Sequence
  HA-SP-3F-GtCLD
MAYPYDVPDYANNNDLFQASRRRFLAQLGGLTVAGMLGPSLLTPRRATAAQADYKDDDDKDYKDDDDKDYKDDDDKGSGPTGMSGPQGLPGPTGPAGQNGSPGSPGAMGPQGPMGPRGYRGLRGPPGDQGRPGDTGRPGAPGIPGQIGQRGPIGPMGVQGPEGPRGNPGLPGPPGAPGVPGIGIQGPAGPPGPPGRF

SEQ ID NO: 87 - protein, 172 aa, Artificial Sequence
  HA-SP-3F-FX-APRO-F-ELP4
MAYPYDVPDYANNNDLFQASRRRFLAQLGGLTVAGMLGPSLLTPRRATAAQADYKDDDDKDYKDDDDKDYKDDDDKGSIEGRRPDFCLEPPYTGPCKARIIRYFYNAKAGLCQTFVYGGCRAKRNNFKSAEDCMRTCGGADYKDDDDKVGVAPGVGVAPGVGVAPGVGVAPG

SEQ ID NO: 88 - protein, 67 aa, Artificial Sequence
  3F-1F-ELP4
DYKDDDDKDYKDDDDKDYKDDDDKGSDYKDDDDKVGVAPGVGVAPGVGVAPGVGVAPGYPYDVPDYA

SEQ ID NO: 89 - protein, 193 aa, Artificial Sequence
  HA-SP-3F-FX-APRO-LGM-TV-ELPE4
MAYPYDVPDYANNNDLFQASRRRFLAQLGGLTVAGMLGPSLLTPRRATAAQADYKDDDDKDYKDDDDKDYKDDDDKGSIEGRRPDFCLEPPYTGPCKARIIRYFYNAKAGLCQTFVYGGCRAKRNNFKSAEDCMRTCGGARSGGGGSSGGGGGGSSRSENLYFQGVGVAPGEVGVAPGEVGVAPGEVGVAPGE

SEQ ID NO: 90 - protein, 191 aa, Artificial Sequence
  HA-SP-3F-FX-APRO-LGM-EK-ELPE4
MAYPYDVPDYANNNDLFQASRRRFLAQLGGLTVAGMLGPSLLTPRRATAAQADYKDDDDKDYKDDDDKDYKDDDDKGSIEGRRPDFCLEPPYTGPCKARIIRYFYNAKAGLCQTFVYGGCRAKRNNFKSAEDCMRTCGGARSGGGGSSGGGGGGSSRSDDDDKVGVAPGEVGVAPGEVGVAPGEVGVAPGE

SEQ ID NO: 91 - protein, 191 aa, Artificial Sequence; identical to SEQ ID NO: 90
  HA-SP-3F-FX-APRO-LGM-EK-ELPE4

(19) United States
(12) Patent Application Publication
(10) Pub. No.: US 2023/0093611 A1
(43) Pub. Date: Mar. 23, 2023

(54) Recombinant microalgae able to produce peptides, polypeptides or proteins of collagen, elastin and their derivatives in the chloroplast of microalgae and associated method thereof

(71) Applicant: ALGANELLE, BOURGET DU LAC (FR)

(72) Inventor: Ghislaine TISSOT-LECUELLE, LA MOTTE SERVOLEX (FR)

(21) Appl. No.: 17/802,560

(22) PCT Filed: Feb. 26, 2021
(86) PCT No.: PCT/EP2021/054928

§ 371 Date: Aug. 26, 2022

(30) Foreign Application Priority Data
Feb. 28, 2020 (EP) 20305210.5

(51) Int. Cl. C12N 15/82

(52) U.S. Cl. CPC C12N 15/8257

(57) ABSTRACT

The present invention concerns a recombinant microalgae comprising a nucleic acid sequence encoding a recombinant protein, polypeptide or peptide comprising repeat units of amino acids, said recombinant protein, polypeptide or peptide being chosen from collagen, elastin and their derivatives, and said nucleic acid sequence being located in the chloroplast of microalgae. It further relates to a method for producing a recombinant protein, polypeptide or peptide comprising repeat units of amino acids in the chloroplast of microalgae, said recombinant protein, polypeptide or peptide being chosen from collagen, elastin and their derivatives wherein said method comprises transforming the chloroplast genome of a microalgae with a nucleic acid sequence encoding said recombinant protein, polypeptide or peptide.

The present invention concerns a recombinant microalgae comprising a nucleic acid sequence encoding a recombinant protein, polypeptide or peptide comprising repeat units of amino acids, said recombinant protein, polypeptide or peptide being chosen from collagen, elastin and their derivatives, said nucleic acid sequence being located in the chloroplast genome of microalgae. It further relates to a method for producing a recombinant protein, polypeptide or peptide comprising repeat units of amino acids in the chloroplast of microalgae, said recombinant protein, polypeptide or peptide being chosen from collagen, elastin and their derivatives wherein said method comprises the chloroplast genome transformation of a microalgae with a nucleic acid sequence encoding said recombinant protein, polypeptide or peptide.

In recent years, the demand for recombinant protein is increasing more and more because of their high value applications in broad range industries as in personal care, cosmetics, healthcare, tissue engineering, biomaterials, agriculture and paper industries. Numerous examples of commercial pharmaceutical proteins produced in various recombinant systems have been launched, as for instance, insulin, human growth hormone, erythropoietin and interferon.

Additionally, large quantities of proteins and peptides are needed for these various industrial applications.

The most current industrial expression systems include the bacteria E. coli, the yeast (S. cerevisiae and P. pastoris) and mammalian cell lines. Emerging technologies are insect cell cultures, plants and microalgae.

However, the expression of recombinant peptides and proteins is still limited, as large efforts are required in order to obtain the desired peptides and proteins with a native conformation, in high amounts and high purity. Even a current bacterial system such as E. coli has limitations at expressing recombinant peptides/polypeptides/proteins. Indeed, formation of insoluble aggregates (or inclusion body) arises due to the lack of sophisticated machinery to perform posttranslational modifications, as for instance disulfide bond formation or glycosylations. This results in poor solubility of the protein of interest and/or in the absence of protein activity.

Interest in microalgae as an alternative platform for recombinant protein production has been gaining in recent years.

Recombinant algae offer several advantages over the other recombinant protein production platforms. Microalgae are photosynthetic unicellular microorganisms with low nutriment requirements to grow. They are capable of photoautotrophic, mixotrophic or heterotrophic growth. The cost of protein production in algae is much lower than other production systems in photoauxotrophic growth. Proteins purified from algae, as from plant, should be free from toxins and viral agents that may be present in preparations from bacteria or mammalian cell culture. Indeed, several microalgae species have the GRAS status (Generally Recognized As Safe) Granted by the FDA, as for instance for microalgae, Chlorella vulgaris, Chlorella protothecoides S106, Dunaliella bardawil, Chlamydomonas reinhardtii and for cyanobacteria Arthrospira plantesis.

As in transgenic plants, algae have been engineered, to express recombinant genes from both the nuclear and chloroplast genomes.

In addition, recombinant synthesis of peptides and polypeptides composed of repeat units of specific amino acid sequence is difficult because the DNA sequences encoding the peptides or polypeptides are often subject to genetic recombination resulting in genetic instability and leading often to the production of proteins smaller than the native one.

Recombinant production of relatively small peptides can be also challengeous because they can self-assemble or be subject to proteolytic degradation.

Moreover, in contrast to plant expression system, algae are robust industrial chassis with competitive production costs reachable at industrial scale in reproducible, sterile and well-controlled production conditions within photobioreactors and fermenters or in a single use wave bag. In addition, they can secrete recombinant proteins outside the cell and thus in the culture media, simplifying the subsequent purification steps. Algae have no seasonality and don't used arable land.

The development of chloroplast transformation in algae for the production of proteins of interest is more recent than in plants and requires improvement. In fact, the production yield of recombinant protein are typically between 0.5 and 5% of the total soluble proteins in Chlamydomonas reinhardtii chloroplast, which is still low in comparison to established microbial platform.

In addition, some mammalian proteins are not easily expressed (Rasala et al., 2010).

The inventors of the present invention have surprisingly been able to produce, in the chloroplast of microalgae, a recombinant protein, polypeptide or peptide comprising several repeat units of amino acids, said recombinant protein, polypeptide or peptide being chosen from collagen, elastin and their derivatives.

Indeed, homologous recombination phenomenon between similar or identical sequences being highly efficiency in the chloroplast genome, transgene with repeat sequences could have been very instable.

By using said method, endogenous disulfide bond formation which is essential for protein, peptide and polypeptide stability and activity, and increased accumulation of the protein, peptide and polypeptide, is allowed.

The present invention thus relates to a recombinant microalgae comprising a nucleic acid sequence encoding a recombinant protein, polypeptide or peptide comprising repeat units of amino acids, said protein, polypeptide or peptide being chosen from collagen, elastin and their derivatives, and said nucleic acid sequence being located in the chloroplast genome of microalgae.

The present invention also relates to the use of said recombinant algae, for producing a recombinant protein, polypeptide or peptide comprising repeat units of amino acids, said protein, polypeptide or peptide being chosen from collagen, elastin and their derivatives.

The present invention further relates to a method for producing a recombinant protein, polypeptide or peptide comprising repeat units of amino acids in the chloroplast of microalgae, said recombinant protein, polypeptide or peptide being chosen from collagen, elastin and their derivatives, wherein said method comprises transforming the chloroplast genome of microalgae with a nucleic acid sequence encoding said recombinant protein, polypeptide or peptide.

In particular, said method comprises:

i) providing a nucleic acid sequence encoding said recombinant protein, polypeptide or peptide;

(ii) introducing the nucleic acid sequence according to (i) into an expression vector which is capable of expressing the nucleic acid sequence in microalgae host cell; and

(iii) transforming the chloroplast genome of microalgae host cell by the expression vector.

In particular, said method further comprises:

(iv) identifying the transformed microalgae host cell;

(v) characterizing the microalgae host cell for the production of recombinant protein, polypeptide or peptide expressed from said nucleic acid sequence;

(vi) extracting the recombinant protein, polypeptide, or peptide; and optionally

(vii) purifying the recombinant protein, polypeptide, or peptide.

More particularly, the method according to the invention allows to increase accumulation and/or stability and/or solubility and/or folding and/or activity of recombinant peptides, polypeptides or proteins comprising repeat units of amino acids in the chloroplast of microalgae.

In particular, said method further comprises a step (viii) further to step (vii) in which the polypeptide is cleaved to allow the release of peptide units.

Said cleavage can be carried out by any method known by the man skilled in the art such as the use of suitable endoproteinase.

In one embodiment, the recombinant protein, polypeptide or peptide obtained by the method according to the invention are chemically modified at their N- or C-terminus, for example by adding a palmitoyl group, an hydroxyl group, an alkoyl chain (i.e. an alkyl chain comprising an hydroxyl group), or a biotinyl group.

By “recombinant peptide/polypeptide/protein” is meant in the art, and in the context of the present invention, an exogenous peptide/polypeptide/protein expressed from a recombinant gene (or recombinant nucleic acid sequence) i.e. an exogenous gene (or exogenous nucleic acid sequence) being from a different species (heterologous) or from the same species (homologous).

By “recombinant microalgae” is meant a microalgae comprising a nucleic acid sequence encoding a recombinant protein, polypeptide or peptide. In the context of the invention, the recombinant microalgae is transformed as further detailed below.

By “peptides”, “polypeptides”, “proteins” is meant the meaning commonly understood by a person skilled in the art to which this invention belongs. In particular, peptides, polypeptides and proteins are amino acid polymers linked via peptide (amide) bonds.

More particularly, proteins according to the invention have unique and stable three-dimensional structure and are composed of more than 50 amino acids, like proteins of 54, 60, 66, 72, 75, 78, 84, 90, 96, 100, 102, 108, 114, 120, 150, 180, 200, 300, 350 or more amino acids; peptides according to the invention are of short oligopeptides, for examples peptides of 2 to 10 amino acids, like peptides of 4, 5, 6, 7, 8, 9 or 10 amino acids; polypeptides according to classical meaning can be composed of 11 to 50 amino acids, like polypeptides of 11, 12, 15, 18, 20, 24, 25, 30, 35, 36, 40, 42, 45, 48, or 50 amino acids; but in the context of the invention, polypeptides are a repetition of n units of identical or different amino acid sequence, or a repetition of n units of identical or different peptide, n being from 2 to 400, in particular from 2 to 100.

In the context of the invention, a recombinant protein, polypeptide or peptide according to the invention is a recombinant protein, polypeptide or peptide chosen from collagen, elastin and their derivatives, comprising repeat units of amino acids.

Elastin is a major structural protein of the extracellular matrix. It is present in the connective tissue of all vertebrates providing elasticity to tissues. Elastin is firstly synthetized as soluble monomer precursors, tropoelastin, which is subsequently assembled into the mature elastin, a stable polymeric structure.

This protein is well known in the art. The amino acid sequence of elastin and tropoelastin contains short repetitive amino acid motifs and numerous hydrophobic residues. During the aging process or after exposure to UVB-irradiation or in pathological processes, elastin is degraded into short peptides, named “elastin peptides” which play the role of signal peptides promoting for instance cell proliferation. These elastin peptides are part of matricins peptides.

Elastin peptides are considered as building blocks found in the natural elastin and have a short amino acid sequence.

Examples of elastin peptides according to the invention are pentapeptides: KGGVG (SEQ ID N^{o} 1), VGGVG (SEQ ID N^{o} 2), GVGVP (SEQ ID N^{o} 3), VPGXG (X being V, I, or K) (SEQ ID N^{o} 4), hexapeptides: VGVAPG (SEQ ID N^{o} 5), heptapeptides: LGAGGAG (SEQ ID N^{o} 6) or nonapeptides: LGAGGAGVL (SEQ ID N^{o} 7).

In the context of the invention, a recombinant peptide, polypeptide or protein consisting in repeated units of elastin, consists in repeated units (identical or different) of peptides of elastin and in particular of those (identical or different) of SEQ ID N^{o} 1 to 7. “Derivatives” of elastin protein/polypeptide/peptide covers both elastin like proteins/polypeptides/peptides and proteins/polypeptides/peptides in which the amino acid sequence of the native elastin protein/polypeptide/peptide is mutated or contains one amino acid or more at its N- or C-terminus. The supplementary amino acids can be any amino acids. Derivatives of elastin protein/polypeptide/peptide also covers derivatives of elastin like proteins/polypeptides/peptides. “Derivatives” of elastin like proteins/polypeptides/peptides covers proteins/polypeptides/peptides in which the amino acid sequence of the native elastin like protein/polypeptide/peptide is mutated or contains one amino acid or more at its N- or C-terminus.

The peptides, polypeptides and peptides of elastin and their derivatives, according to the invention, thus also cover elastin like peptides, polypeptides, peptides and their derivatives.

Elastin like proteins, polypeptides or peptides (ELP) are synthetic molecules comprising mainly several fold repeated units of peptides or derivatives.

By “mutated” peptide, polypeptide or protein is meant that the nucleic or amino acid sequences of the mutated peptide, polypeptide or protein contains one or more mutations. These mutations include deletions, substitutions, insertions and/or cleavage of one or more nucleic acids or amino acids.

There are many variants of elastin-like polypeptides, comprising repeated units of different elastin peptides, such as those of SEQ ID N^{o} 1 to 7.

The elastin like polypeptides or proteins described in this invention can thus, for example, comprise a n-fold repeat of pentapeptides: (KGGVG)ₙ, (VGGVG)ₙ, (GVGVP)ₙ, (VPGXG)ₙ, hexapeptides: (VGVAPG)ₙ, heptapeptides: (LGAGGAG)ₙ or nonapeptides: (LGAGGAGVL)n, or of their derivatives. The number n can be for example from 2 to 200, with a preference from 2 to 100.

As an example, the elastin peptide described in this invention is the hexapeptide of SEQ ID N^{o} 5 (VGVAPG) or its derivatives.

The elastin like polypeptides or proteins described in this invention can thus, for example, comprise a n-fold repeat of this hexapeptide: (VGVAPG)ₙ, or of its derivatives. The number n can be for example from 2 to 200, with a preference from 2 to 100.

In particular, derivatives of the peptide of SEQ ID N^{o} 5 contain the VGVAPG sequence with one or more amino acids at its N- and/or C-terminus.

In particular, the supplementary amino acids in the derivatives of the peptide of SEQ ID N^{o} 5 are an aspartic acid or a glutamic acid.

More particularly, said derivatives can be of SEQ ID N^{o} 8 (VGVAPGD) or SEQ ID N^{o} 9 (VGVAPGE).

Still particularly the elastin like polypeptide described in this invention comprises a repeat of the hexapeptide of SEQ ID N^{o} 5, more particularly a 4-fold of this hexapeptide (named in the present invention ELP4 and being of SEQ ID N^{o} 81: VGVAPGVGVAPGVGVAPGVGVAPG). Another example of elastin like polypeptide derivative described in this invention comprises a repeat of the hexapeptide of SEQ ID N^{o} 9, more particularly a 4-fold of this hexapeptide (named in the present invention ELPE4 and being of SEQ ID N^{o} 80: VGVAPGEVGVAPGEVGVAPGEVGVAPGE).

An interesting feature of ELPs is that they can self-aggregates with an increase of temperature or a modification in pH and ionic strength. This feature can facilitated the purification step of ELPs after production in host cells.

The collagens are a superfamily of structurally related proteins that constitute essential building elements of connective tissues and participate to many biological functions in animals. These proteins exhibit a characteristic triple-helix tertiary structure resulting from the association of three polypeptide chains comprising repeated amino acid sequence Gly-X-Y (or GXY) where the amino acids X and Y are frequently proline or 4-hydroxyproline involved in the triple helix formation.

In humans, there are at least 27 different types of collagens found in different tissues (as bones, bones, skin, tendon, blood vessels, eyes, etc. . . . ).

In the context of the invention, a recombinant peptide, polypeptide or protein of collagen consists in repeated units of collagen motifs, i.e. in repeated units of the sequence GXY.

“Derivatives” of collagen protein/polypeptide/peptide covers both collagen like proteins/polypeptides/peptides and proteins/polypeptides/peptides in which the amino acid sequence of the native collagen protein/polypeptide/peptide is mutated or contains one amino acid or more at its N- or C-terminus. This supplementary amino-acids can be any amino acids. Derivatives of collagen protein/polypeptide/peptide also covers derivatives of collagen like proteins/polypeptides/peptides. “Derivatives” of collagen like protein/polypeptide/peptide covers proteins/polypeptides/peptides in which the amino acid sequence of the native collagen like protein/polypeptide/peptide is mutated or contains one amino acid or more at its N- or C-terminus. This supplementary amino acids can be any amino acids.

Collagen like proteins, peptides or polypeptides comprise mainly several fold repeated units of collagen motifs, i.e. of repeated units of the sequence GXY (whereas peptide, polypeptide or protein of collagen contain only those repeated units).

Said repeated units are called “collagen like domain” and are capable to form triple helix (as collagen proteins), as for instance, C-type lectins (collectins), which participate in the host defense mechanism.

Moreover, screening of genome databases of gene encoding collagen-like sequences containing repeated GXY motifs have identified gene in the genomes of bacteria and phages. However, these organisms seem to lack proline hydroxylases. Recent studies have shown that two recently identified streptococcal collagen-like proteins, Scl1 and Scl2 as models are capable of forming a stable triple helix without hydroxylation of the proline residues.

In particular, collagen like proteins, peptides or polypeptides are and/or derive (of one or more amino acids) in particular in the present invention from microalgae, as for instance, from the CCMP2712 protein from Guillardia theta (called GtCLP).

In one embodiment, derivatives according to the invention consists in an amino acid sequence at least 80% identical to the amino acid sequence of the recombinant peptide, polypeptide or protein according to the invention.

By “an amino acid sequence at least 80% identical” is meant in particular, an amino acid sequence 81, 82, 85, 90, 91, 92, 93, 94, 95, 96, 97, 98 or 99% identical. By an amino acid sequence at least 95% “identical” to a query amino acid sequence of the present invention, it is intended that the amino acid sequence of the subject peptide, polypeptide or protein is identical to the query sequence except that the subject amino acid sequence may include up to five amino acids alterations per each 100 amino acids of the query amino acid sequence. In other words, to obtain an amino acid sequence at least 95% identical to a query amino acid sequence, up to 5% (5 of 100) of the amino acid residues in the subject sequence may be inserted, deleted, or substituted with another amino acid.

In the frame of the present application, the percentage of identity is calculated using a global alignment (i.e. the two sequences are compared over their entire length). Methods for comparing the identity of two or more sequences are well known in the art. The “needle” program, which uses the Needleman-Wunsch global alignment algorithm (Needleman and Wunsch (1970) J. Mol. Biol. 48:443-453) to find the optimum alignment (including gaps) of two sequences when considering their entire length, may for example be used. The needle program is for example available on the ebi.ac.uk world wide web site. The percentage of identity in accordance with the invention is preferably calculated using the EMBOSS::needle (global) program with a “Gap Open” parameter equal to 10.0, a “Gap Extend” parameter equal to 0.5, and a Blosum62 matrix.

Amino acid sequences “at least 80%, 85%, 90%, 95% or 99% identical” to a reference sequence may comprise mutations such as deletions, insertions and/or substitutions compared to the reference sequence. In case of substitutions, amino acid sequences at least 80%, 85%, 90%, 95% or 99% identical to a reference sequence may correspond to a homologous sequence derived from another species than the reference sequence. In another preferred embodiment, the substitution preferably corresponds to a conservative substitution as indicated in the table below.

| Conservative substitutions | Type of Amino Acid |
|---|---|
| Ala, Val, Leu, Ile, Met, Pro, Phe, Trp | Amino acids with aliphatic hydrophobic side chains |
| Ser, Tyr, Asn, Gln, Cys | Amino acids with uncharged but polar side chains |
| Asp, Glu | Amino acids with acidic side chains |
| Lys, Arg, His | Amino acids with basic side chains |
| Gly | Neutral side chain |

According to the invention, a nucleic acid sequence encoding said recombinant protein, polypeptide or peptide is a nucleic acid sequence encoding the proteins, peptides or polypeptides mentioned above.

In particular, for proteins, a nucleic acid sequence encoding elastin protein, elastin like proteins, collagen or collagen like proteins is contemplated, in particular, a collagen like protein is contemplated.

For example, the following nucleic acid sequence encoding a collagen like protein can be cited, 3f-tv-Gtclp-tv-ha (SEQ ID N^{o} 10).

In particular, for peptides, a nucleic acid sequence encoding collagen like peptides, collagen peptides, elastin peptides and elastin like peptides is contemplated; more particularly a nucleic acid sequence encoding a collagen like peptide or elastin like peptide.

For example, the following nucleic acid sequences can be cited: SEQ ID N^{o} 11 (GTAGGTGTAGCTCCTGGT), SEQ ID N^{o} 12 (GTTGGTGTTGCTCCTGGA), SEQ ID N^{o} 13 (GTAGGTGTTGCTCCAGGT) and SEQ ID N^{o} 14 (GTGGGTGTAGCTCCTGGT), all these nucleic sequences encoding the elastin peptide VGVAPG previously mentioned. Other nucleic acid sequences can be cited as examples such as SEQ ID N^{o} 15 (GTAGGTGTAGCTCCTGGTGAA), SEQ ID N^{o} 16 (GTTGGTGTTGCTCCTGGAGAA), SEQ ID N^{o} 17 (GTAGGTGTTGCTCCAGGTGAA) and SEQ ID N^{o} 18 (GTGGGTGTAGCTCCTGGTGAA), all encoding the elastin peptide derivative VGVAPGE previously mentioned.

In particular, for polypeptides, a nucleic acid sequence encoding collagen like polypeptides, collagen polypeptides, elastin polypeptides and elastin like polypeptides are contemplated; more particularly a nucleic acid sequence encoding collagen polypeptide or an elastin like polypeptide.

For example, the following nucleic acid sequences can be cited ha-sp-3f-Gtccld-3ha (SEQ ID N^{o} 19), ha-sp-3f-Gtccld (SEQ ID N^{o} 20) and ha-sp-3f-Gtcld (SEQ ID N^{o} 21), elp4 (SEQ ID N^{o} 22), and elpe4 (SEQ ID N^{o} 23).

Still particularly, nucleic acid sequence encoding derivatives according to the invention consists in a nucleic acid sequence at least 80% identical to the nucleic acid sequence encoding the recombinant peptide, polypeptide or protein according to the invention. By “a nucleic acid sequence at least 80% is meant in particular, a nucleic acid sequence 81, 82, 85, 90, 91, 92, 93, 94, 95, 96, 97, 98 or 99% identical. For example, a nucleic acid sequence 95% “identical” to a query sequence of the present invention, is intended to mean that the sequence of the polynucleotide is identical to the query sequence except that the sequence may include up to five nucleotide alterations per each 100 nucleotides of the query sequence. In other words, to obtain a polynucleotide having a sequence at least 95% identical to a query sequence, up to 5% (5 of 100) of the nucleotides of the sequence may be inserted, deleted, or substituted with another nucleotide. In other terms, the sequences should be compared on their entire length (i.e. by preparing a global alignment). For example, a first polynucleotide of 100 nt (nucleotides) that is comprised within a second polynucleotide of 200 nt is 50% identical to said second polynucleotide. The needle program, which uses the Needleman-Wunsch global alignment algorithm (Needleman and Wunsch (1970), A general method applicable to the search for similarities in the amino acid sequence of two proteins, J. Mol. Biol. 48:443-453) to find the optimum alignment (including gaps) of two sequences when considering their entire length, may for example be used. Preferably, the percentage of identity in accordance with the invention is calculated using the needle program with a “Gap open” parameter equal to 10.0, a “Gap Extend” parameter equal to 0.5, and a Blosum 62 matrix. The needle program is for example available on the ebi.ac.uk World Wide Web site.

In one embodiment, the nucleic acid sequence encoding the protein, polypeptide or peptide comprising repeat units of amino acids according to the invention is codon optimized for expression in the chloroplast genome of the microalgae host cell.

As mentioned above, the nucleic acid sequence according to the invention is introduced into an expression vector which is capable of expressing the nucleic acid sequence.

By “introduction” is meant cloning the nucleic acid sequence encoding the recombinant protein/polypeptide/peptide inside the expression vector with the methods well known by the skilled man and in the way to lead to the expression of this nucleic acid sequence.

“Expression vector” or “transformation vector” or “recombinant DNA construct”, or similar terms, are defined herein as DNA sequences that are required for the transcription of recombinant genes and the translation of their mRNAs in the microalgae host cells. “Expression vector” comprises one or more expression cassettes for the expression of recombinant genes (one or more genes encoding the protein, peptide or polypeptide of interest and often selectable markers). In the case of chloroplast genome transformation, expression vectors also contain homologous recombination regions for the integration of expression cassettes inside the chloroplast genome.

In the context of the invention, the expression vector can be in particular a circular molecule with a plasmid backbone containing the two homologous recombination regions and flanking the expressions cassettes, or a linearized molecule corresponding to the expression vector linearized by enzymatic digestion or to a PCR fragment containing only the expression cassettes flanked by the two homologous recombination regions.

In particular, expression vectors of the invention comprise at least one expression cassette and are for example vectors pCO86, pCO96, pCO26, pCO28, pLA01, pLA02, pAL03 or pAL04.

“Expression cassette” contains a coding sequence fused operationally to one or more regulatory elements or regulatory sequences, as for instance, fused at its 5′end to a promoter and/or 5′UTR and/or at its 3′end to a 3′UTR.

The “coding sequence” is the portion of a gene and of its corresponding transcribed mRNA which is translated into the recombinant protein/polypeptide/peptide. The coding sequence includes, for example and a translation initiation control sequence and a stop codon. In some embodiment, the expression cassette can contain a polycistron composed of more than one coding sequence encoding several proteins under the control of only one promoter/5′UTR and 3′UTR.

Said expression cassettes are flanked by left (LHRR) and right (RHRR) endogenous sequences identical to those surrounding the targeted integration site into the chloroplast genome. These left (LHRR) and right (RHRR) homologous regions allow the integration of expression cassettes after homologous recombination exchange between the regions of homology.

Homologous recombination is the ability of complementary DNA sequences to align and exchange regions of homology. Transgenic DNA (“donor”) containing sequences homologous to the genomic sequences being targeted (“template”) is introduced into the organism and then undergoes recombination into the genome at the site of the corresponding genomic homologous sequences.

By its very nature homologous recombination is a precise gene targeting event, hence, most transgenic lines generated with the same targeting sequence will be essentially identical in terms of phenotype, necessitating the screening of far fewer transformation events.

In the case of chloroplast genome transformation of microalgae, the integration of expression cassettes inside the chloroplast genome occurs after homologous recombination between the endogenous homologous sequences of the expression vector with the genome sequences identical or similar to those surrounding the targeted integration site into the chloroplast genome.

In the context of the present invention, different integration sites can be used, between the genes rbcL and atpA, or psaB and trnG, or atpB and 16S rDNA, or psaA exon3 and trnE, or trnE and psbH or psbN and psbT, or psbB and trnD.

In some embodiments, in order to enhance its accumulation, the recombinant protein or polypeptide or peptide can be fused to endogenous proteins, as for instance to the large subunit of ribulose bisphosphate carboxylase (Rubisco LSU). In this case, the promoter and 5′UTR will be those of the endogen rbcL gene, after homologous recombination of the transformation vector into the chloroplast genome.

The protein, peptide or polypeptide of interest will be further separated from RBCL either in vivo (using self-cleavable peptides) or in vitro (by site specific proteolysis), depending of the chosen processing system.

In one embodiment, said coding sequence of the expression cassette according to the invention also comprises a nucleic acid sequence encoding an epitope tag, in particular the Flag epitope Tag, more particularly the Flag epitope Tag repeat 3 times (3×Flag Tag), in order to identify and/or purify the recombinant protein, polypeptide or peptide.

In particular, said epitope Tag sequence is placed at the N-terminus of the protein, peptide or polypeptide. More particularly, another epitope Tag sequence can be placed at the C-terminus of the protein, peptide, or polypeptide according to the invention, alone or in addition to the one at the N-terminus and this, in order to monitor the release of the peptide/polypeptide/protein of interest to follow its cleavage, for example by an endoprotease.

Examples of epitope Tag sequences are Flag Tag (SEQ ID N^{o} 24: DYKDDDDK), 3×Flag Tag (SEQ ID N^{o} 25: DYKDDDDKDYKDDDDKDYKDDDDK), HA Tag (SEQ ID N^{o} 26: YPYDVPDYA), 3×HA Tag (SEQ ID N^{o} 27: YPYDVPDYAYPYDVPDYAYPYDVPDYA), His Tag (SEQ ID N^{o} 28: HHHHHH), which are described in the experimental part of the invention.

In one embodiment, said coding sequence in the expression cassettes comprises nucleic acid sequences encoding not only the said recombinant protein, polypeptide or peptide but also an amino acid sequence allowing the production of the said recombinant protein, polypeptide or peptide in specific cell compartment.

“Promoter” as used herein, refers to a nucleic acid control sequence that directs transcription of a nucleic acid.

“5′UTR” or 5′ untranslated region (also known as a leader sequence or leader RNA) is the region of an mRNA that is directly upstream from the initiation codon.

“3′UTR” or 3′ untranslated region is the section of messenger RNA (mRNA) that immediately follows the translation termination codon.

5′UTRs and 3′UTRs are required for transcript (mRNA) stability and translation initiation.

For microalgae chloroplast expression, promoters, 5′UTRs and 3′UTRs that can be used in the context of the invention are for example: the promoters and 5′UTRs of the genes psbD, psbA, psaA, atpA, and atpB, the 16S rRNA promoter (Prrn) promoter fused with a 5′UTR, the psbA 3′ UTR, the atpA 3′UTR or the rbcL 3′ UTR.

A 5′UTR from exogenous origin as for instance the 5′UTR of the gene 10L of the bacteriophage T7 can be used also fused downstream a microalgae promoter. In particular, the nucleic acid sequence is operationally linked at its 5′end to the Chlamydomonas reinhardtii 16S rRNA promoter (Prrn).

Stable expression and translation of the nucleic acid sequence according to the present invention can for example be controlled by the promoter and 5′UTR from psbD and the atpA 3′UTR.

Also, in one embodiment of the recombinant microalgae or method according to the invention, nucleic acid sequence encoding a recombinant protein, polypeptide or peptide is operably linked to at least one regulatory sequence chosen from the psbD promoter and 5′UTR (SEQ ID N^{o} 29), or the 16S rRNA promoter (Prrn) promoter fused with the atpA 5′UTR (SEQ ID N^{o} 30), the psaA promoter and 5′UTR, the atpA promoter and 5′ UTR, the 3′UTRs from atpA (SEQ ID N^{o} 31) and rbcL (SEQ ID N^{o} 32).

In one embodiment, the promoter less gene encoding the protein, peptide or polypeptide of interest can be integrated after homologous recombination region inside the chloroplast genome just downstream a native promoter.

As mentioned above, the chloroplast genome of microalgae host cell is transformed by the expression vector. This genetic transformation of microalgae host cells, and more particularly the chloroplast genome of microalgae, by expression vector according to the invention can be carried out according to any suitable techniques well known by the man skilled in the art including, without limitations biolistics (Boynton et al., 1988; Goldschmidt-Clermont, 1991), electroporation (Fromm et al. (1985) Proc. Natl. Acad. Sci. (USA) 82:5824-5828; see Maruyama et al. (2004), Biotechnology Techniques 8:821-826), glass bead transformation (Purton et al., revue), protoplasts treated with CaCl₂) and polyethylene glycol (PEG) (see Kim et al. (2002) Mar. Biotechnol. 4:63-73) or microinjection.

In particular, said transformation uses the helium gun bombardment technique of gold micro-projectiles complexed with transforming DNA.

To identify the microalgal transformants, a selectable marker gene may be used. Mention may be made for example of the aadA gene coding aminoglycoside 3″-adenylyltransferase and conferring the resistance to spectinomycin and streptomycin in the case of Chlamydomonas reinhardtii chloroplast transformation. In another embodiment, the selectable marker gene can be the Acinetobacter baumannii aphA-6 Ab gene encoding 3′-aminoglycoside phosphotransferase type VI and conferring the kanamycin resistance.

Chloroplast genome engineering can thus be performed using selectable maker gene conferring resistance to antibiotic or using rescue of photosynthetic mutant.

In particular, in one embodiment, the expression vector for chloroplastic genome transformation comprises two expression cassettes comprising the nucleic acid sequence encoding the recombinant protein, polypeptide or peptide according to the invention or the selectable marker gene.

More particularly, the expression vector for chloroplastic genome transformation comprises one expression cassette comprising the nucleic acid sequence encoding the recombinant protein, polypeptide or peptide according to the invention and one expression cassette comprising the aadA gene coding aminoglycoside 3″-adenylyltransferase.

In another embodiment of the invention, corresponding to the case of rescue of photosynthetic mutant sensitive to light, the expression vector comprises the wild type RHRR region which is deleted in the mutant strain. After homologous recombinations, the deleted region is restored in the genome of the photosynthetic mutant which is able then to growth under light.

In particular, the coding sequence of the expression cassette according to the invention also comprises a nucleic acid sequence encoding a signal peptide. By “signal peptide” (SP) is meant in the present invention an amino acid sequence placed at the N-terminus of a newly synthetized recombinant protein or polypeptide or peptide. This signal peptide should allow the translocation of the protein inside the lumen of chloroplast thylakoids and not in the chloroplast stroma. The signal peptide is cleaved after translocation across the thylakoid membranes.

For example, such signal peptide sequence is chosen from known protein translocated inside the lumen of thylakoids using either the twin-arginine protein translocation (Tat) pathway or the Sec pathway.

For instance, signal peptides can derive from algae proteins localized in the thylakoid lumen, as the signal peptide from the Chlamydomonas reinhardtii 16 and 23 kDa subunits of the oxygen-evolving complex of photosystem II, or the Chlamydomonas reinhardtii Rieske subunit of b₆f complex or the cryptophytes phycoerythrin alpha subunit (as example from Guillardia theta).

In particular, the Signal Peptide can be extracted from the sequence of the E. coli TorA gene encoding the Trimethylamine-N-oxide reductase 1 (UniProt number P33225) (SP; SEQ ID N^{o} 33: NNNDLFQASRRRFLAQLGGLTVAGMLGPSLLTPRRATAAQA; nucleic acid sequence encoding SP is SEQ ID N^{o} 34). This signal peptide used the Tat system. This amino acid sequence is cleaved from the protein after the translocation of the later one across the thylakoid membrane.

Other signal peptides can be used which don't leave supplementary amino acids at the N-terminus of the recombinant protein as in particular signal peptide from algae, and in particular from Chlamydomonas reinhardtii.

In one embodiment, the recombinant protein or polypeptide or peptide can be produced as a fusion protein.

The invention thus also relates to a recombinant microalgae or method according to the invention, wherein the nucleic acid sequence encoding a recombinant protein, polypeptide or peptide is fused operationally at its 5′ or 3′end to a nucleic acid sequence encoding a carrier.

Fusion partners or carriers have been developed in recombinant protein production in order to increase accumulation yields, and/or solubility and/or folding and/or to facilitate protein purification. Fusion partners of different sizes (or molecular weight) have been used in various production systems in order to enhance protein solubility and accumulation (maltose-binding protein (MBP), glutathione-S-transferase (GST), thioredoxin, GB1, N-utilizing substance A (NusA), ubiquitin, small ubiquitin-like modifier (SUMO), Fh8) and to facilitate detection and purification (as for examples without limitation MBP, GST and small epitope Tag peptides as c-myc Tag, poly-histidine Tag (His Tag), Flag Tag, HA Tag. Another type of fusion Tags used for purification are stimulus response Tags (or environmentally responsive polypeptides) which allow precipitation of the fusion protein when stimulus as modification of temperature or solution ionic strength are adjusted.

In particular, a carrier according to the present invention is aprotinin.

The carrier and the recombinant protein, polypeptide or peptide are fused together to form a fusion protein.

By «aprotinin» is meant the basic trypsin inhibitor (BPTI), a small single-chain protein cross-linked by three disulfide bridges which consists of 58 amino acid residues with a molecular mass of 6.5 kDa and an isoelectric point of 10.9.

Said protein is well known by the man skilled in the art and is available commercially. It can for example be produced in recombinant systems such as plants (in cytoplasm by nuclear transformation (Pogue et al., 2010), or in thylakoid lumens by chloroplast transformation (Tissot et al., 2008).

Its formula is C₂₈₄H₄₃₂N₈₄O₇₉S₇ and its molar mass 6511.51 g/mol.

The amino acid sequence for aprotinin from Bos Taurus (bovine) is RPDFC LEPPY TGPCK ARIIR YFYNA KAGLC QTFVY GGCRA KRNNF KSAED CMRTC GGA (SEQ ID N^{o} 35). The nucleic acid sequence encoding this amino acid sequence is the SEQ ID N^{o} 36.

In the context of the present invention, the term “aprotinin” also covers chimeric aprotinin and mutated aprotinin.

By “chimeric aprotinin” is meant that aprotinin is connected at its N-terminus and/or at its C-terminus to an epitope Tag peptide(s) and/or signal peptide and/or protease recognition cleavage site.

The chimeric aprotinin can be for example the protein called HA-APRO (SEQ ID N^{o} 37), comprising aprotinin fused at its N-terminus to the HA epitope Tag or 3 F-APRO (SEQ ID N^{o} 39) comprising aprotinin fused at its N-terminus to the 3×Flag epitope Tag (3F).

Other examples of chimeric aprotinin can be the protein called HA-SP-3F-FX-APRO (SEQ ID N^{o} 41 and 42) comprising aprotinin fused at its N-terminus to an amino acid sequence made of the HA epitope Tag (HA) followed by the signal peptide (SP), the 3×Flag epitope Tag (3F), and the cleavage site for Factor Xa (FX; IEGR), or the chimeric aprotinin called HA-SP-3F-APRO (SEQ ID N^{o} 43 and 44) comprising aprotinin fused at its N-terminus to the HA epitope Tag followed by the signal peptide SP and the 3×Flag epitope Tag (3F) or the chimeric aprotinin called HA-SP-APRO (SEQ ID N^{o} 45 and 46) comprising aprotinin fused at its N-terminus to the HA epitope Tag followed by the signal peptide SP.

By “mutated” aprotinin is meant that the nucleic or amino acid sequence of the “mutated” aprotinin contains one or more mutations in the nucleic or amino acid sequences of aprotinin or chimeric aprotinin. These mutations include deletions, substitutions, insertions and/or cleavage of one or more nucleic acids or amino acids.

Signal peptide is as previously described.

Other signal peptides can be used which doesn't leave supplementary amino acids at the N-terminus of the recombinant protein. If the signal peptide is cleaved after translocation into the lumen of chloroplast thyalkoids (or across the thylakoid membranes), two other chimeric aprotinin can be produced in vivo 3F-APRO or 3F-FX-APRO (SEQ ID N^{o} 47).

In particular, according to the invention, the fusion partner is used to improve the accumulation and/or the stability of recombinant peptides, polypeptides and proteins.

Still particularly, and as mentioned above, said fusion protein also comprises cleavage sites recognized by a specific protease.

Cleavage sites recognized by specific proteases are well known of the man skilled in the art. They are used to separate the aprotinin from the recombinant protein, polypeptide or peptide of interest, in the case the carrier should be removed if it could interfere with the activity or the structure of said protein, polypeptide or peptide and thus with its uses.

In particular, said cleavage sites are an endoprotease and/or endoproteinase recognition sequence (or protease cleavage site or protease recognition site). More particularly, the sequence of said cleavage sites is placed between the two coding sequences (the one of aprotinin and the one of the recombinant protein, polypeptide or peptide of interest according to the invention).

The cleavage of the fusion protein can be performed either in vivo (in the recombinant host cell before extraction or when apply on the skin for cosmetic peptides) or in vitro after extraction and purification by adding protease.

Non limitative examples of proteases are Factor Xa (FX), Tobacco Edge Virus protease (TEV), enterokinase (EK), SUMO protease, Thrombin, Human Rhinovirus 3C Protease (HRV 3C), endoproteinase Arg-C, endoproteinase Asp-C, endoproteinase Asp-N, endoproteinase Lys-C, endoproteinase Glu-C, proteinase K, IgA-Protease, Trypsin, chymotrypsin and Thermolysin.

Self-cleavage peptides can also be used, as for example the Intein system (Yang et al., 2003), the viral 2A system (Rasala et al., 2012) or the site of the preferredoxin from Chlamydomonas (Muto at al., 2009).

In one embodiment, a linker can be placed between aprotinin and the protease cleavage site. Linkers can be classified into three types: flexible, rigid and cleavable. The usual function of linkers is to fuse the two partners of the fusion protein (e.g. flexible linkers or rigid linkers) or to release them under specific conditions (cleavable linkers) or to provide other functions of the proteins in drug design such as improving of their biological activities or their targeted delivery.

In one embodiment of the present invention, the linker can also make the protease cleaving site more accessible to the enzyme if necessary.

In one embodiment, the flexible linker contains small, non-polar (e.g. Gly) or polar (e.g. Ser or Thr) amino acids. Examples of such linkers are given in Chen et al., 2013.

Flexible linkers according to the invention can be LG (SEQ ID N^{o} 49: RSGGGGSGGGGSGS) or LGM (SEQ ID N^{o} 50: RSGGGGSSGGGGGGSSRS).

When a fusion protein with a carrier is involved, step (vii) of the method according to the invention is a step of purification the fusion protein.

In that case, the method comprises optionally a step (viii) in which the fusion protein is cleaved.

Said cleavage can be carried out by any method known by the man skilled in the art such as the use of suitable protease to release the recombinant peptide, polypeptide or protein.

Said step (viii) is optionally followed by a purification step (ix) of the recombinant protein, polypeptide or peptide.

In particular, said method further comprises a step (viii′) between step (viii) and step (ix), in which the polypeptide is cleaved to allow the release of peptide units.

Said cleavage can be carried out by any method known by the man skilled in the art such as the use of suitable endoproteinase Characterization of the microalgae host cell producing the recombinant protein, polypeptide or peptide can be conducted by techniques known by the man skilled in the art, for example by PCR screening of the antibiotic resistant transformants or Western Blot analysis performed on total protein extracts.

Extraction of total proteins can be carried out using well known techniques (centrifugation, lysis, sonication, etc).

Identification of fusion or recombinant proteins can be carried out by Western Blot using specific antibodies.

Purification can be carried out using well-known techniques. In one embodiment, it comprises an affinity chromatography and/or a step of separation of the peptide, polypeptide or protein according to the invention from the carrier (for example by enterokinase protease digestion) and/or a size exclusion chromatography.

In one embodiment, the step of affinity chromatography can be replaced by an ion exchange chromatography, less expensive for large scale purification.

According to the invention, “microalgae” is a eukarytotic microbial organism that contains a chloroplast or plastid, and optionally that is capable of performing photosynthesis, or a prokaryotic microbial organism (cyanobacteria) capable of performing photosynthesis.

In particular, said microalgae is chosen from the group consisting Chlorophyta (green algae), Rhodophyta (red algae), Stramenopiles (heterokonts), Xanthophyceae (yellow-green algae), Glaucocystophyceae (g laucocystophytes), Chlorarachniophyceae (chlorarachniophytes), Euglenida (eug len ids), Haptophyceae (coccolithophorids), Chrysophyceae (golden algae), Cryptophyta (cryptomonads), Dinophyceae (dinoflagellates), Haptophyceae (coccolithophorids), Bacillariophyta (diatoms), Eustigmatophyceae (eustigmatophytes), Raphidophyceae (raphidophytes), Scenedesmaceae, Phaeophyceae (brown algae).

More particularly, said microalgae is chosen from the group consisting of Chlamydomonas, Chlorella, Dunaliella, Haematococcus, diatoms, Scenedesmaceae, Tetraselmis, Ostreococcus, Porphyridium, and Nannochloropsis.

Even more particularly said microalgae is chosen from the group consisting of Chlamydomonas, more particularly Chlamydomonas reinhardtii, even more particularly Chlamydomonas reinhardtii 137c or a deficient strain as Chlamydomonas reinhardtii CW15.

In particular, said microalgae is cultured in classic conditions known by the man skilled in the art. For example, Chlamydomonas reinhardtii is grown in TAP (Tris Acetate Phosphate) medium to mid-logarithmic phase (densities of approximately 1-2×10⁶ cell/ml), and/or at a temperature comprised between 23° C. to 25° C. (ideally 25° C.), and/or on a rotary shaker in presence of constant light (70-150 μE/m²/s). The experimental part illustrates the conditions of culture.

All the embodiments mentioned in the context of the present invention can be combined.

The invention will be further illustrated by the following figures and examples.

### FIGURES

FIG. 1: Codon usage in the Chlamydomonas reinhardtii chloroplast genome

FIG. 2: Schematic presentation of the chloroplast transformation vectors for the production of G. theta CCMP2712 protein (GtCLP), the native and chimeric Collagen-Like Domains (GtCLD) of G. theta CCMP2712.

FIG. 3: Western Blot analysis of independent algae transformants CW-CO86 and CW-CO96 expressing the genes of G. theta CCMP2712 protein (A) and the chimeric Collagen-Like domain of G. theta CCMP2712 (B) from the algae chloroplast genome, using monoclonal anti-Flag M2 antibody. 100 μg of each total soluble protein samples extracted with SDS buffer lysis from the wild type (WT) CW15 and from the transformants CW-CO86 and CW-CO96 were separated on a 15% SDS polyacrylamide gel. MW: molecular weight standard. Arrows indicate the positions of recombinant proteins.

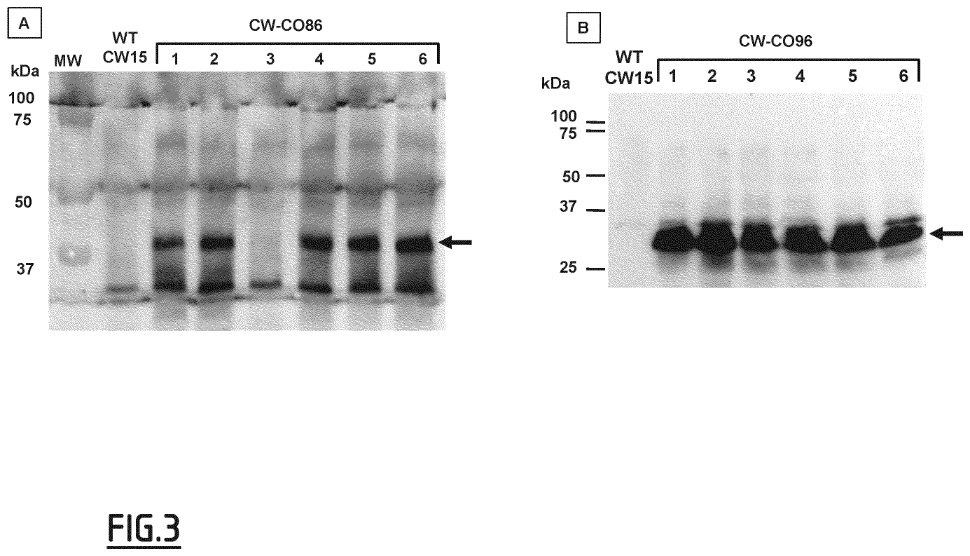

FIG. 4: Western Blot analysis of 137c-CO96-4 transformant using monoclonal anti-Flag M2 (A) or anti-HA (B) antibodies. 50 μg of total soluble protein from 137c-CO96 transformants extracted by sonication were digested (+EK; A: Lane 4; B: Lane 5) or not (A: Lane 2 and 3; B: 3 and 4) by enterokinase (EK). 50 μg of total soluble protein from wild type (WT) 137c extracted with SDS buffer lysis. MW: molecular weight standard. Arrows indicate the positions of recombinant proteins with or without the Flag Tag.

FIG. 5: Western Blot analysis of different elution fractions from anti-HA affinity chromatography performed on a protein extract from 137c-CO96-4 and using monoclonal anti-HA antibody. Protein samples (50 μg of protein extracted by sonication wild type (WT) 137c or 25 μl of elution or wash fractions) were loaded on a 15% SDS polyacrylamide gel. MW: molecular weight standard. Load: total soluble protein extracted by sonication before the incubation with anti-HA resin. FT: Flow through. EA: elution fraction. W: wash fraction. Arrows indicate the positions of purified recombinant proteins.

FIG. 6: Schematic presentation of the chloroplast transformation vectors for elastin polypeptide and peptide production.

FIG. 7: Western Blot analysis of algae cells transformed with pLA01, using monoclonal anti-Flag (A) or anti-HA (B) antibodies. 50 μg of total soluble protein samples extracted with SDS buffer lysis from wild type (WT) CW15 cells and from independent CW-LA01 transformants were separated on a 15% SDS polyacrylamide gel. MW: molecular weight standard. Arrows indicate the positions of recombinant proteins.

### EXAMPLES

### Example 1

Material and Methods

All oligonucleotides and synthetic genes were purchased from Eurof ins. All enzymes were purchased from NEB, Promega, Invitrogen and Sigma Aldrich/Merck. All plasmids were built on the pBluescript II backbone.

### Algal Strains and Growth Conditions

The two algal strains used are the Chlamydomonas reinhardtii wild type (137c; mt+) and the cell wall deficient strain CW15 (CC-400; mt+), obtained from the Chlamydomonas Resource Center, University of Minnesota).

Prior to transformation, all strains were grown in TAP (Tris Acetate Phosphate) medium to mid-logarithmic phase (densities of approximately 1-2×10⁶ cell/mL) at a temperature comprised between 23° C. to 25° C. (ideally 25° C.) on a rotary shaker in presence of constant light (70-150 μE/m²/s).

Transformants were grown in the same conditions and the same media containing 100 μg/mL of spectinomycin or 100 μg/mL kanamycin, depending of the selectable marker gene present in the transformation vector.

Growth kinetics was also followed by measuring the optical density at 750 nm using a spectrophotometer.

### Algal Transformation

Chlamydomonas reinhardtii cells were transformed using the helium gun bombardment technique of gold micro-projectiles complexed with transforming DNA, as described in the article Boynton et al., 1988. Briefly, the Chlamydomonas reinhardtii cells were cultivated in TAP medium until midlog phase, harvested by gentle centrifugation, and then resuspended in TAP medium to a final concentration of 1.10⁸ cells/mL. 300 μL of this cell suspension was plated onto a TAP agar medium supplemented with 100 μg/mL of spectinomycin or 100 μg/mL of kanamycin, depending of the selectable marker gene present in the transformation vector. The plates were bombarded with gold particles (S550d; Seashell Technology) coated with transformation vector, as described by the manufacturer. The plates were then placed at 25° C. under standard light conditions to allow selection and formation of transformed colonies.

### Total DNA Extraction and PCR Screening of Positive Transformants

Total DNA extraction was performed using the chelating resin Chelex 100 (Biorad) from single colonies (with size of around 1 mm in diameter) of wild type and/or antibiotic resistant transformants Chlamydomonas strains.

From isolated colonies, a quantity of cells corresponding to about 0.5 mm in diameter was removed with a pick and resuspended in 20 μL of H₂O. 200 μL of ethanol were added and incubated 1 min at room temperature. 200 μL of 5% Chelex were incorporated and vortexed. After an incubation of 8 min at 100° C., the mixture was cooled down and centrifuged 5 min at 13,000 rpm. Finally, the supernatant was collected.

After transformation, algae colonies growing onto restrictive solid medium plates were expected to have the antibiotic resistant gene and the other transgene(s) incorporated the transgene(s) into their genome.

In order to identify stable integration of the recombinant genes into the algal genome, the antibiotic resistant transformants were screened by Polymerase Chain Reaction (PCR or PCR amplification) in a thermocycler using 1 μL of total DNA previously extracted as template, two synthetic and specific oligonucleotides (primers) and Taq polymerase (GoTaq, Promega). The cycles of PCR amplification followed the guidelines recommended by the manufacturer. The PCR reactions were subjected to gel electrophoresis in order to check the PCR fragment of interest.

### Protein Extraction, Western Blot Analyses

Chlamydomonas cells (50 mL, 1-2.10⁸ cells/mL) were collected by centrifugation. Cell pellet was resuspended in lysis buffer (50 mM Tris-HCl pH 6.8, 2% SDS, 10 mM EDTA). As further detailed below, in some embodiments of the example, the lysis buffer didn't contain 10 mM EDTA. After 30 min at room temperature, cell debris were removed by centrifugation at 13000 rpm and the supernatant containing the total soluble proteins was collected.

Depending on the further analysis step, total soluble proteins were extracted under non denaturing conditions in different buffers. Cell pellet was resuspended in a buffer containing 50 mM Tris-HCl (pH 6.8 or 8) or 20 mM Tris-HCl (pH 6.8 or 8). The sonication step was carried out with the algal cell suspension held on ice, using a cell disruptor a sonicator FB505 500W (Sonic/FisherBrand) and a setting of the micro-tip probe to 20% power, with continuous sonication for 5 min. After sonication, cell debris were removed by centrifugation at 13000 rpm, 30 min.

Total soluble proteins present in the supernatant were quantified using the Pierce BCA protein assay kit, following the instructions of the supplier (Thermofisher).

Total soluble protein samples (50 or 100 μg or another quantity further mentioned in the example depending of the experiment) were separated in a 12 or 15% Tris-glycine SDS-PAGE prepared according to Laemmli (1970).

For experiments performed under reducing conditions, samples were prepared in Laemmli sample loading buffer with 50 mM DTT (or more depending of the fusion protein) or 5% Beta-mercaptoethanol, and further denaturated 5 min at 95° C. before loading. The SDS PAGE experiments were carried out using a Protein Gel tank from BioRad.

After separation, samples were blotted onto a nitrocellulose membrane (GE HealthCare) using standard transfer buffer and a Trans-Blot® Turbo™ Transfer System from Biorad. In order to visualize the transferred proteins, the nitrocellulose membrane were stained by Ponceau S dye. Membranes were further blocked with Tris-buffered saline Tween buffer (TBS-T) (50 mM Tris-HCl pH 7.5, 150 mM NaCl, 0.1% Tween-20) containing 5% Bovin Serum Albumin (BSA). After one hour of saturation at room temperature under gently shaking, membranes were incubated during one night at 4° C. with TTBS buffer containing mouse primary antibody (See table 1).

TABLE 1
Primary antibodies

| Primary antibody | Source | Dilution |
|---|---|---|
| Monoclonal ANTI-FLAG ® M2 antibody | Sigma | 1:1000 |
| produced in mouse | | |
| Monoclonal ANTI-HA PURIFIED antiobody | Sigma | 1:6000 |
| produced in mouse IGG | | |
| Monoclonal ANTI-Aprotinin antibody | Abeam | 1:2000 |
| produced in mouse | | or |
| | | 1:3000 |

After three washes with TBS-T-BSA buffer, membranes were incubated one hour at room temperature with TBS-T-BSA buffer containing secondary antibodies (Anti-Mouse IgG (H+L), HRP Conjugate; Promega). After four washes with TTBS buffer and one wash with TBS buffer, the membranes were incubated in an enhanced chemiluminescence (ECL) substrate (Clarity Max ECL substrate; Biorad). The ECL signals were visualized with the ChemiDoc™ XRS+ system (Biorad).

### Protein Purification

After centrifugation, algae cell pellets were resuspended in different buffers depending on the protein and on the further steps to which the protein is submitted. If the next step was an anti-FLAG M2 affinity chromatography, the buffer contained 50 mM Tris-HCl pH8, 500 mM NaCl and 0.1% Tween 20. If the next step was an anti-HA affinity chromatography, the buffer contained 20 mM Tris-HCl pH8. Approximately, 10 mL of buffer were used per g of wet algal cells, depending of the transformants. The resuspended cells were sonicated in the same conditions as previously described.

### Affinity Chromatography

All recombinant proteins were tagged in their N-terminal with a Flag Tag epitope which will bind specifically on an anti-Flag M2 affinity gel (Sigma/Merck). This resin contains a mouse monoclonal Anti-Flag® M2 antibody that is covalently attached to agarose.

All steps of this experiment were carried out as described by the manufacturer. Briefly, the samples of total soluble proteins were filtered using a cellulose acetate 0.45 μm filter and mixed with anti-Flag® M2 affinity gel prepared as recommended by the manufacturer and equilibrated in binding buffer (50 mM Tris-HCl pH8, 500 mM NaCl, 0.1% Tween 20). Approximately, 1 mL of resin was used per 4 to 8 g of wet algal cells, depending of the transformants. Binding of the recombinant fusion protein was performed at 4° C. for 4 h or overnight with a gently and continuous end-over-end mixing. After incubation, the mixture of soluble protein incubated with resin were loaded by gravity on an empty Bio-rad Econo-pac column or collected by centrifugation, and washed several times with 40 column volumes of TBST and 20 column volumes of TBS. The protein of interest was eluted from the resin using 100 mM Glycine pH 3.5, 500 mM NaCl and neutralized with Tris-HCl pH 8 to a final concentration of 50 mM.

Some recombinant proteins were tagged with a HA epitope Tag which will bind specifically on an anti-HA agarose resin (Pierce/Thermo Scientific). All steps of this experiment were carried out as described by the manufacturer. Briefly, the filtered samples of total soluble proteins were mixed with an anti-HA agarose resin prepared as recommended by the manufacturer and equilibrated in TBS and incubated overnight at 4° C. with a gently and continuous end-over-end mixing or a rocking platform. After incubation, the resin was pelleted 5 to 10 second at 12,000 g (repeat 3 times). The supernatant was kept for further analysis. The pelleted resin was washed several times with 10 bed volumes of TBST. The protein of interest was eluted from the resin after incubation 15 min at 30° C. of the resin with 10 bed volumes of 1 mg/ml Pierce HA peptide. The resin was pelleted by centrifugation (5 to 10 second at 12,000 g). The supernatant containing the fusion protein was collected. This elution step was repeat 3 additional times.

Each elution fractions of affinity chromatography were further analyzed by SDS-PAGE and Western Blot.

Depending of the further step, the elution fractions containing the protein of interest were dialyzed in Slide-A-Lyzer Dialysis Cassettes (3.5 kDa MWCO, Thermo Scientific) as described by the manufacturer against the buffer used in the further step, as for instance, for the protease digestion. The dialyzed samples were concentrated using Vivaspin 6 (3 kDa MWCO, GE Healthcare).

Separation of the Protein of Interest from the Carrier

The separation of the protein of interest from the carrier was made by protease digestion, in particular, in the present invention by enterokinase (light chain) or Tobacco Etch Virus (TEV) Protease from New England BioLabs (NEB).

Enzymatic digestions were performed as recommended by the manufacturer.

For example, for enterokinase light chain digestion, reactions combined 25 μg of protein of interest in 20 μL of buffer (20 mM Tris-HCl pH 8.0, 50 mM NaCl, 2 mM CaCl₂)), with 1 μL of enterokinase light chain. Incubation was made at 25° C. for 16 h.

For example, for TEV digestion, typical reaction recommended by the manufacturer combined 15 μg of protein substrate with 5 μL of TEV protease reaction buffer (10×) to make a 50 μL total reaction volume. After addition of 1 μL of TEV Protease, reaction was incubated at 30° C. for 1 hour or 4° C. overnight.

For example, for Factor Xa digestion, the manufacturer recommended to digest 50 μg of fusion protein with 1 μg of FXa in a volume of 50 μL at 23° C. for 6 h. The reaction buffer consisted in 20 mM Tris-HCl pH 8.0, 100 mM NaCl and 2 mM CaCl₂).

### Cleavage of the Polypeptide by Endoproteinase

The choice of the endoproteinase used to cleave the polypeptide of interest depends of the amino acid sequence of this polypeptide. Endoproteinases can be for instance, endoproteinase Glu-C, endoproteinase Arg-C, endoproteinase Asp-C, endoproteinase Asp-N, or endoproteinase Lys-C.

Enzymatic digestions were performed as recommended by the manufacturer. For example, for endoproteinase Glu-C digestion (from NEB), the manufacturer recommended to digest 1 μg of substrate protein with 50 ng of endoproteinase Glu-C at 37° C. for 16 h. The reaction buffer consisted in 50 mM Tris-HCl pH 8.0 and 0.5 mM GluC-GluC.

### Size Exclusion Chromatography (SEC)

Size-exclusion chromatography of purified and digested fusion protein was performed using an AKTA Pure system (GE Healthcare) in order to separate the protein of interest from the carrier.

A Superdex S30 Increase G10/300 GL column (GE Healthcare) and a HiLoad 26/600 Superdex 30 prep grade column were first calibrated using two standards diluted with 2×PBS buffer (or appropriate buffer for the further step): aprotinin (bovine lung; 6.5 kDa), and glycine (75 Da).

After a washing step in water, the Superdex S30 Increase G10/300 GL column was equilibrated in running buffer (2×PBS, pH 7.4, or 1×PBS, pH 7.4 or appropriate buffer for the further step) and 200 to 500 μL samples were run through the column at a rate of 0.5 mL/min. Elution of protein was detected by measuring optical absorbance at 280, 224 and 214 nm. 0.5 mL fractions were collected and analyzed by SDS-PAGE followed by Western Blot or stained by Coomassie Blue dye.

After a washing step in water, the HiLoad 26/600 Superdex 30 prep grade column was equilibrated in running buffer (2×PBS, pH 7.4, or 1×PBS, pH 7.4 or the appropriate buffer for the further step) and samples (4 to 30 mL) were run through the column at a rate of 2.6 mL/min. Elution of proteins was detected by measuring optical absorbance at 280, 224 and 214 nm. 4 mL fractions were collected and analyzed by SDS-PAGE followed by Western Blot.

In some embodiment, the elution fractions of interest were pooled and evaporated using a SpeedVac (Eppendorf). The peptides or polypeptides or proteins present in these evaporated samples were subjected to Edman degradation to confirm the amino acid sequence at the N-terminus of the protein of interest.

### Example 2

Production of Collagen Like Protein, Collagen Like Domain and Collagen Like Polypeptide in the Chloroplast of Chlamydomonas reinhardtii by Chloroplast Genome Transformation

### Construction of Transformation Vector for the Expression of the Collagen-Like Protein

In order to produce an innovating collagen-like protein (CLP) and/or collagen-like domain, we screened databases to find collagen-like genes encoding collagen-like domain from different origins. One of the sequence founded was the CCMP2712 protein from the microalgae Guillardia theta (G. theta). The amino acid sequence of the CCMP2712 protein from G. theta (called GtCLP SEQ ID N^{o} 51) contained collagen like domain and was extracted from GenBank Accession Number XM 005827950.

Nothing was described about the capability of the protein encoded by the identified gene to form a collagen-like triple-helical structure.

In Chlamydomonas reinhardtii, the codon usage has been shown to play a significant role in protein accumulation (Franklin et al., 2002; Mayfield and Schultz, 2004).

The nucleic acid sequence encoding CCMP2712 protein was designed and optimized in order to improve its expression in C. reinhardtii host cell. Methods for altering nucleic acid sequence for improved expression in host cell are known in the art, particularly in algae cell, particularly in C. reinhardtii.

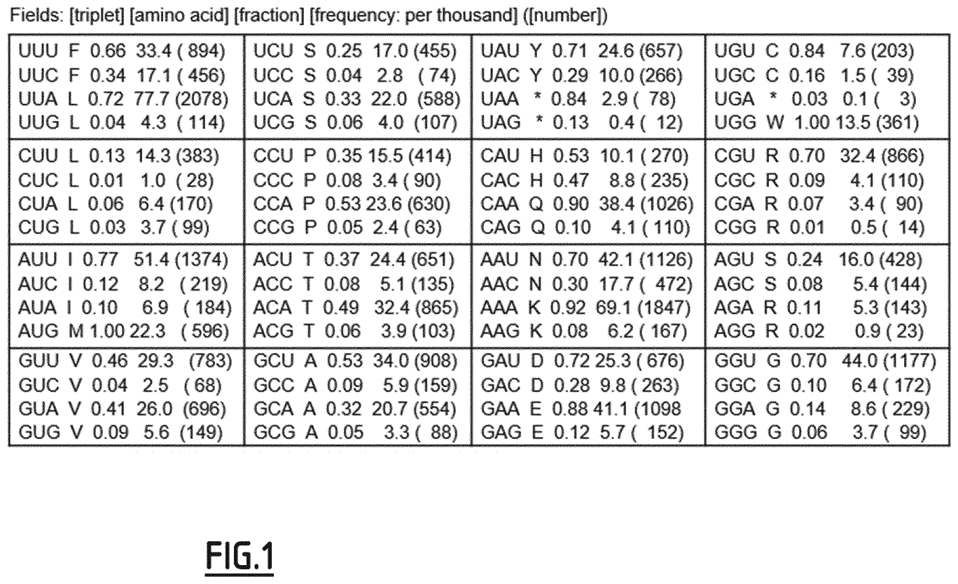

A codon usage database was found at http://www.kazusa.or.jp/codon/ (See the codon usage for chloroplast genome of C. reinhardtii; FIG. 1).

For improving expression in C. reinhardtii chloroplast of the gene of interest, codons from their native sequence which are not commonly used, were replaced with a codon coding for the same or a similar amino acid residue that is more commonly used in the C. reinhardtii chloroplast codon bias. In addition, other codons were replaced to avoid sequences of multiple or extended codon repeats, or some restriction enzyme site, or having a higher probability of secondary structure that could reduce or interfere with expression efficiency.

In order to check and to fulfill all criteria mentioned above, the amino acid sequence of the protein of interest were also optimized by the software GENEius of Eurofins using the appropriate codon usage for C. reinhardtii chloroplast.

After its codon optimisation, the gene Gtclp encoding the native CCMP2712 protein from G. theta (called “recombinant GtCLP” or 3F-TV-GtCLP-TV-HA) was designed to be operationally fused at its 5′end to the codon optimized nucleic acid sequences encoding an amino acid sequence containing the 3×Flag epitope Tag (SEQ. DYKDDDDKDYKDDDDKDYKDDDDK; SEQ ID N^{o} 25) followed by the recognition site of the TEV protease (SEQ ENLYFQG; SEQ ID N^{o} 52). At its 3′end, the optimized gene Gtclp were operationally fused to the optimized nucleic acid sequence coding for the recognition site of the TEV protease followed by the HA epitope Tag (SEQ ID N^{o} 26). The recombinant GtCLP produced in vivo in C. reinhardtii chloroplast was also called 3F-TV-GtCLP-TV-HA. The two recognition sites of the TEV protease allowed the elimination of the 3×Flag and HA Tags by in vitro protease digestions.

This resulting fusion gene 3f-tv-Gtclp-tv-ha (SEQ ID N^{o} 10) encoding the recombinant GtCLP called 3F-TV-GtCLP-TV-HA (SEQ ID N^{o} 55) was synthesized and cloned by Eurofins Genomics into the vector pEX-A258 resulting in vector pAL70.

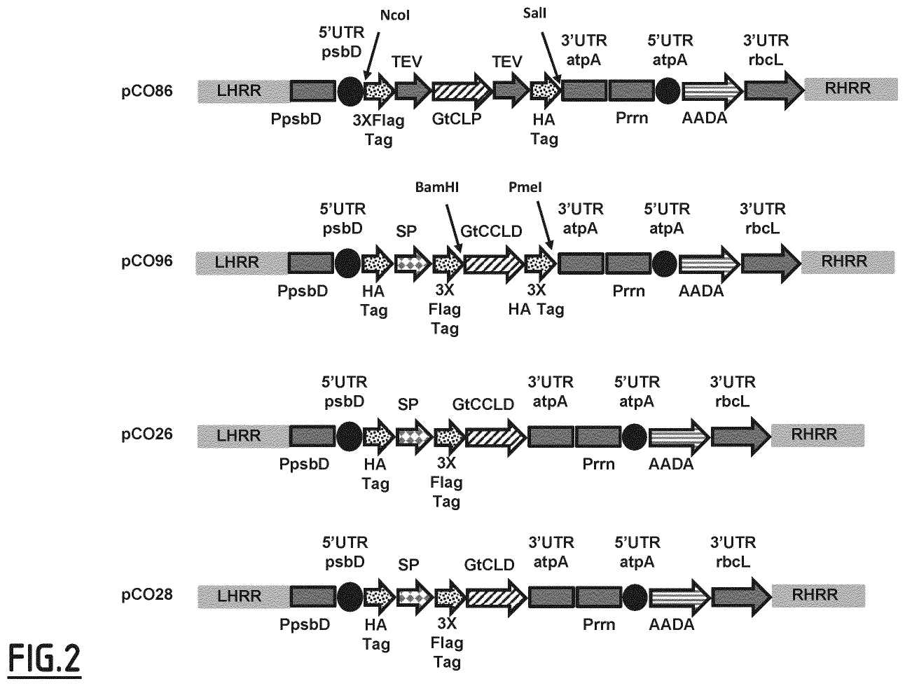

After PCR amplification from the vector pAL70 using the primers O5′SCL70 (SEQ ID N^{o} 56) and O3′SCL70 (SEQ ID N^{o} 57), the PCR fragment FPCR-SCL70 of 1317 bp (SEQ ID N^{o} 58) was cloned using the Gibson Assembly of New England Biolabs (as recommended by the manufacturer) into the expression cassette of the gene of interest (goi) present in the chloroplast transformation vector pLE56 linearized by NcoI and SalI to form the vector pCO86 (FIG. 2).

The chloroplast expression vector pLE56 contained two expression cassettes for the expression of the genes encoding the selectable marker (gos) and the recombinant protein of interest (goi). The selection cassette contained the selectable marker aadA gene coding aminoglycoside 3″-adenylyltransferase and conferring the resistance to spectinomycin and streptomycin. This gene was operationally linked at its 5′ end to the C. reinhardtii 16S rRNA promoter (Prrn) fused to the atpA 5′UTR and at its 3′end to the 3′UTR of the C. reinhardtii rbcL gene. In the second cassette, stable expression of the recombinant goi was controlled by the promoter and 5′UTR from the C. reinhardtii psbD and the 3′UTR from the C. reinhardtii atpA.

These two expression cassettes were flanked by a left (LHRR) and right (RHRR) endogenous homologous recombination sequences which were identical to those surrounding the targeted integration site into the C. reinhardtii chloroplast genome. The choice of the insertion site within the chloroplast genome was generally made such as not to disrupt an essential gene or interrupt the expression of a polycistronic unit. In a preferred embodiment, the chloroplast transformation vectors in the present invention allowed the targeted integration of the transgenes into the chloroplast genome of C. reinhardtii between the 5S rDNA and psbA genes (and derives from instance from the sequence GenBank Accession Number NC005352).

Construction of Chloroplast Transformation Vector for the Expression of the Chimeric Collagen-Like Domain of G. theta CCMP2712 CLP.

The collagen-like domain from G. theta CCMP2712 protein (SEQ ID N^{o} 59; named GtCLD) was designed in order to fuse at its N-terminus with an amino acid sequence containing a Cys Knot and CR4 Repeat sequences (SEQ. GPCCGPPGPPGPPGPP, SEQ ID N^{o} 60). The Cys Knot and CR4 Repeat sequences were known in the art as adding conformational rigidity of sequence. At its C-terminus, GtCLD was fused with a CR4 Repeat followed by Cys Knot sequences and the Foldon fibritin of the T4 phage (SEQ. GYIPEAPRDGQAYVRKDGEWVLLSTFL, SEQ ID N^{o} 61). The resulting recombinant protein was named chimeric collagen-like domain from G. theta or GtCCLD (SEQ ID N^{o} 62). The GtCCLD protein was also designed to be fused at its C-terminus a 3×HA epitope Tag (called 3HA; SEQ ID N^{o} 63: YPYDVPDYAYPYDVPDYAYPYDVPDYA).

The synthetic codon optimized gene Gtccld-3ha (SEQ ID N^{o} 84) were synthetized and cloned in the vector pEX-A258 by Eurofins Genomics to form the pAL81. The gene Gtccld-3ha was subcloned in the goi expression cassette of the chloroplast transformation vector pLE63 previously linearized by BamHI and PmeI digestions to give pCO96. More precisely, Gtccld-3ha was subcloned downstream the nucleic acid sequence ha-sp-3F encoding the HA epitope Tag (HA) linked to the signal peptide (SP) followed by the 3×Flag epitope Tag (HA-SP-3F) (FIG. 2).

Then, in C. reinhardtii chloroplast, the recombinant GtCCLD-3HA (SEQ ID N^{o} 64) was produced fused at its N-terminus with the amino acid sequence HA-SP-3F and was called HA-SP-3F-GtCCLD-3HA (SEQ ID N^{o} 83). This recombinant protein was encoded by the nucleic acid sequence called ha-sp-3f-Gtccld-3ha (SEQ ID N^{o} 19). The chloroplast expression vector pLE63 contained the same expression cassette for the selectable marker as pLE56 (FIG. 2). Stable expression of the goi was controlled by the promoter and 5′UTR from the C. reinhardtii psbD and the 3′UTR from the C. reinhardtii atpA. In this expression cassette, the goi was fused at its 5′end to a nucleic acid sequence ha-sp-3F. pLE63 allowed the same targeted integration of the recombinant genes into the C. reinhardtii chloroplast genome as pLE56 and pCO86.

In order to remove the nucleic sequence encoding the 3×HA Tag, the PCR fragment Gtccld-2 (SEQ ID N^{o} 65) containing the nucleic sequence encoding the recombinant chimeric collagen-like domain from the G. theta GtCCLD was amplified by PCR from pAL81 and cloned by the Gibson assembly method between the psbD promoter/5′UTR and the atpA 3′UTR into pLE63 linearized by BamHI and PmeI to form pCO26 (FIG. 2). As in pCO96, the gene cold-2 was subcloned downstream the nucleic acid sequence ha-sp-3F encoding the amino acid sequence HA-SP-3F (FIG. 2).

Then, in C. reinhardtii chloroplast, the recombinant chimeric collagen-like domain from the G. theta GtCCLD was produced fused at its N-terminus with the amino acid sequence HA-SP-3F and was called HA-SP-3F-GtCCLD (SEQ ID N^{o} 85) encoded by the nucleic acid sequence ha-sp-3f-Gtccld (SEQ ID N^{o} 20).

In order to produce only the collagen-like domain from G. theta without Cys Knot and CR4 Repeat sequences, the PCR fragment Gtcld (SEQ ID N^{o} 66) containing the nucleic sequence the gene was amplified by PCR from pAL81 and cloned by the Gibson assembly method between the psbD promoter/5′UTR and the atpA 3′UTR into pLE63 linearized by BamHI and PmeI to form pCO28 (FIG. 2). As in pCO96 and pCO26, the gene Gtcld was subcloned downstream the nucleic acid sequence ha-sp-3F encoding the amino acid sequence HA-SP-3F (FIG. 2).

Then, in the C. reinhardtii chloroplast, the recombinant collagen-like domain from the G. theta GtCLD was produced fused at its N-terminus with the amino acid sequence HA-SP-3F and was called HA-SP-3F-GtCLD (SEQ ID N^{o} 86) encoded by the nucleic acid sequence ha-sp-3f-Gtcld (SEQ ID N^{o} 21).

After their production in algae chloroplast, the signal peptide (SP) will be cleaved in vivo from the recombinant proteins HA-SP-3F-GtCCLD-3HA, HA-SP-3F-GtCCLD and HA-SP-3F-GtCLD during their translocations into the thylakoids. Thus, 3 others proteins could be produced 3F-GtCCLD-3HA, 3F-GtCCLD and 3F-GtCLD.

The 3×Flag Tag will be cleaved by in vitro enterokinase digestion of these recombinant proteins.

### Transformation of Algae

The transformation vectors pCO86, pCO96, pCO26 and pCO28 were bombarded in C. reinhardtii cells (137c and CW15) as described in the example 1.

In order to identify stable integration of the recombinant genes encoding fusion protein into the chloroplast algal genome, spectinomycin resistant colonies were screened by PCR analysis. For positive PCR screens of the fusion protein gene in CO96, CO26 and CO28 transformants, the primers O5′ASTatpA2 (SEQ ID N^{o} 67) and O3′SUTRpsbD (SEQ ID N^{o} 68) annealing, respectively, in the atpA 3′UTR and psbD 5′UTR were used. For CO86 transformants, two other primers were used O5′SCL70 (SEQ ID N^{o} 69) and O5′PpsbDCla2 (SEQ ID N^{o} 70), annealing, respectively, in the gene of interest and psbD promoter.

### Analyses and Results

Western Blot analysis were performed in reducing conditions on total soluble protein samples extracted from several transformants obtained after transformation with pCO86, pCO96, pCO26 and pCO28 (from 137c and CW15 strains).

The results showed that the four recombinant proteins were produced, being probed by the anti-Flag antibody and/or anti-HA antibody.

The FIG. 2 showed the results of Western Blots on total soluble protein extracts from CW-CO86 and CW-CO96 transformants producing the recombinant proteins, 3F-TV-GtCLP-TV-HA and HA-SP-3F-GtCCLD-3HA.

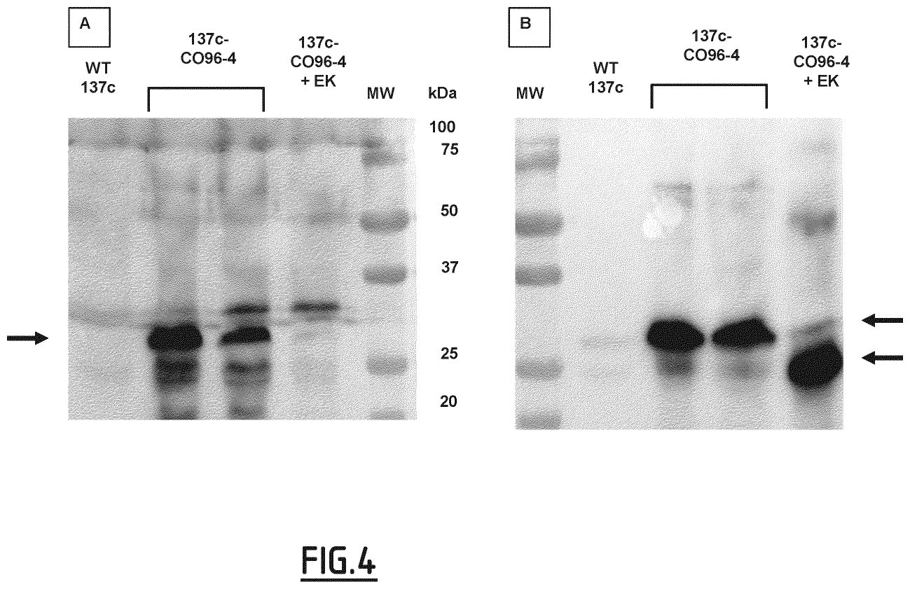

In order to release the recombinant proteins of interest from the N-terminus epitope Tag fused or not with the signal peptide SP, total soluble protein samples extracted from one clone of CO96, CO26 and CO28 transformants were digested in vitro by enterokinase digestion as described in example 1. The FIG. 4 showed the Western Blots performed on the enterokinase digestion of total soluble protein samples extracted from 137c-CO96-4 transformant. The recombinant protein HA-SP-3F-GtCCLD-3HA produced in vivo was cleaved to from GtCCLD-3HA.

In the case of CO86 transformants producing the recombinant protein 3F-TV-GtCLP-TV-HA, total soluble protein samples were digested in vitro either by enterokinase to give the protein TV-GtCLP-TV-HA or by TEV protease to give the protein GtCLP.

137c-CO96-4 cells were produced from around 900 mL cultures. Algae cells (around 3 g) were resuspended and sonicated as described in the example 1. 14.6 mL of total soluble protein extract was obtained.

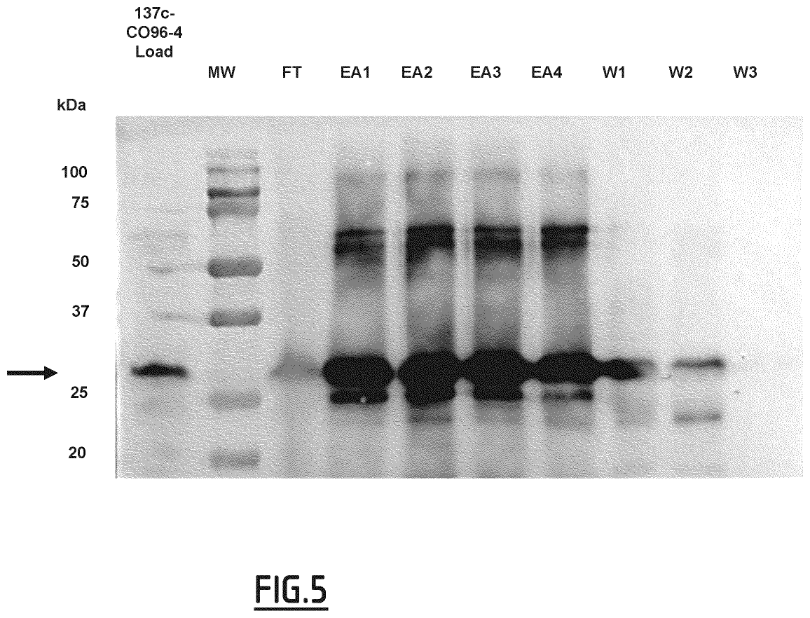

Recombinant protein from 6.5 mL protein extract from 137c-CO96-4 were purified by affinity chromatography using anti-HA resin (150 μl). Elution fractions were analysed by Western Blot analysis. As an example, the results, shown in FIG. 5 revealed the effectiveness of the affinity chromatography purification of the chimeric collagen like domain.

Western Blot analysis performed in non-reducing conditions (without DDT and boiling or not the protein sample before loading onto the polyacrylamide gel) showed that the chimeric collagen like domain produced in CO96 transformants formed in vivo multimeric structures of high apparent molecular weight in contrast to an apparent molecular weight of the protein in reducing conditions.

### Example 3

Production of Elastin Like Peptides or Polypeptides or Derivatives in a Fusion Protein Using Aprotinin as a Carrier in the Chloroplast of Chlamydomonas reinhardtii by Chloroplast Genome Transformation
Construction of Transformation Vectors (pLA01, pLA02, pAL03 and pAL04)

In chloroplast transformation vector, ELP4, an elastin like polypeptide consisted of a repeat of the VGVAPG hexapeptide (SEQ ID N^{o} 5), more particularly of a 4-fold repeat of this hexapeptide (SEQ ID N^{o} 81: VGVAPGVGVAPGVGVAPGVGVAPG), was expressed in a fusion protein in which it was fused, at the C-terminus of the chimeric aprotinin HA-SP-3F-FX-APRO (SEQ ID N^{o} 41 and 42). This fusion partner contained aprotinin fused at its N-terminus to an amino acid sequence made of the HA epitope Tag (HA) followed by the signal peptide (SP), the 3×Flag epitope Tag (3F), and the cleavage site for Factor Xa (FX; SEQ ID N^{o} 71: IEGR). The Flag epitope Tag sequence (SEQ ID N^{o} 24: DYKDDDDK) which is the cleavage site for the enterokinase was inserted between the chimeric aprotinin and ELP4 in order to allow the release of ELP4 from aprotinin by in vitro site specific proteolysis of the fusion protein with enterokinase.

After the production in algae chloroplasts of the fusion protein HA-SP-3F-FX-APRO-F-ELP4 (SEQ ID N^{o} 87), the N-terminus fragment HA-SP will be cleaved during protein translocation into the thylakoids, and the following recombinant protein 3F-FX-APRO-F-ELP4 will be produced in vivo.

As explained in Example 2, in Chlamydomonas reinhardtii, the codon usage in the nucleic acid sequence encoding protein of interest has been shown to play a significant role in protein accumulation.

The nucleic acid sequence encoding the aprotinin were designed and optimized as described in Example 2 in order to improve their expression in C. reinhardtii host cells. After optimization, the gene encoding aprotinin (APRO) were operationally fused at its 5′end to a codon optimized nucleic acid sequences encoding the HA epitope Tag (HA) followed by a signal peptide, the 3×Flag epitope Tag (3F) and the cleavage site recognized by the Factor Xa protease (FX) to form the chimeric aprotinin gene ha-sp-3f-fx-apro (SEQ ID N^{o} 42).

The nucleic acid sequence encoding ELP4 was first codon-optimized using also the same method described in Example 1 and the codon usage of the C. reinhardtii chloroplast genome. The resulting sequence was used to design two overlapping oligomers O5′Gibs-ELP4 (SEQ ID N^{o} 72) and O3′Gibs-ELP4 (SEQ ID N^{o} 73) which were used as primers and template to amplify by PCR the fragment FGibs-ELP4 of 194 bp. This amplified DNA were cloned using the Gibson Assembly Master Mix from New England Biolabs (as recommended by the manufacturer) into the chloroplast transformation vector pAU76 and linearized by PmeI to form the vector pLA00.

The expression vector pAU76 for chloroplast genome transformation contained two expression cassettes for the expression of the genes encoding the selectable marker (identical to previous transformation vector) and the chimeric aprotinin HA-SP-3F-FX-APRO. pAU76 allowed the same targeted integration of the recombinant genes into the C. reinhardtii chloroplast genome as pLE63.

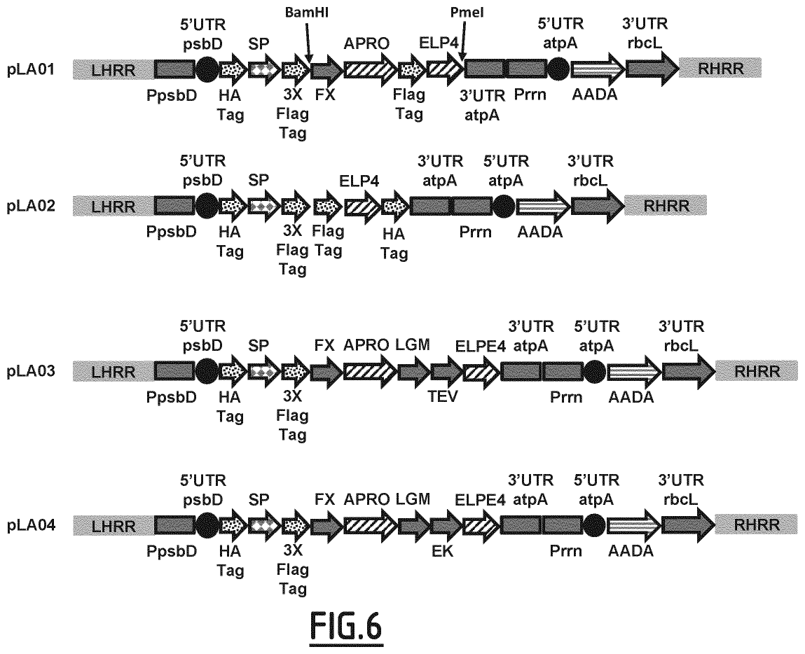

The nucleic acid sequence encoding ELP4 were amplified by PCR from pLA00 using the primers O5′Gibs01BE (SEQ ID N^{o} 74) and O3′Gibs01BE (SEQ ID N^{o} 75). The PCR fragment FPCR-AP-FELP4 (SEQ ID N^{o} 76) of 359 pb were cloned using the Gibson Assembly Master Mix into the pLE63 linearized by BamHI and PmeI to form the vector pLA01. The transformation vector pLA01 allowed the production of the fusion protein HA-SP-3F-FX-APRO-F-ELP4 containing ELP4 linked at its N-terminus to the chimeric aprotinin HA-SP-3F-FX-APRO followed by the 1×Flag Tag (FIG. 6).

The chloroplast transformation vector pLA02 was obtained by cloning by the Gibson Assembly method into pLE63 (linearized by BamHI and PmeI), the PCR fragment FPCR-FELP4-HA (SEQ ID N^{o} 77) (359 pb) amplified from pLA00 with primers O5′Gibs02BE (SEQ ID N^{o} 78) and O3′Gibs02BE (SEQ ID N^{o} 79). The transformation vector pLA02 allowed the production of fusion protein HA-SP-3F-1F-ELP4 containing ELP4 linked at its N-terminus to the chimeric sequence HA-SP-3F followed by the 1×Flag Tag (FIG. 6).

In the case of algae chloroplasts transformed by pLA01 or pLA02 and if the signal peptide SP is cleaved after translocation of the fusion protein into the lumen of thylakoids, two other different proteins can be produced in vivo, 3F-FX-APRO-1F-ELP4 or 3F-1F-ELP4 (SEQ ID N^{o} 88).

In both types of transformation vectors, the release of ELP4 from the fusion proteins can be performed in vitro by enterokinase digestion which cleaved the protein sequence after the second lysine amino acid in the motif sequence DYKDDDDK (SEQ ID N^{o} 24) present in the 1×Flag Tag just upstream ELP4.

The elastin like polypeptide named ELPE4 consisted of a repeat of the VGVAPGE (SEQ ID N^{o} 9), a derivative of the peptide VGVAPG, more particularly of a 4-fold repeat of this peptide (SEQ ID N^{o} 80, VGVAPGEVGVAPGEVGVAPGEVGVAPGE). In chloroplast transformation vector, ELPE4 was also expressed in a fusion protein in which it was fused at the C-terminus of the chimeric aprotinin HA-SP-3F-FX-APRO.

In order to separate in vitro by the ELPE4 from the carrier, a flexible linker LGM (SEQ ID N^{o} 50: RSGGGGSSGGGGGGSSRS) followed by a cleavage site for TEV protease (TV; SEQ ID N^{o} 52: ENLYFQG) or enterokinase (EK; SEQ ID N^{o} 38: DDDDK) were added.

Two types of fusion proteins have been produced from two different chloroplast expression vectors: HA-SP-3F-FX-APRO-LGM-TV-ELPE4 (SEQ ID N^{o} 89) or HA-SP-3F-FX-APRO-LGM-EK-ELPE4 (SEQ ID N^{o} 90).

The nucleic acid sequence encoding LGM-TV-ELPE4 or LGM-EK-ELPE4 were codon-optimized using also the same method described in Example 1 and the codon usage for chloroplast genome of C. reinhardtii. After codon optimization, the different synthetic genes Igm-tv-elpe4 (SEQ ID N^{o} 40) and Igm-ek-elpe4 (SEQ ID N^{o} 48) were synthetized by Eurofins. These optimized genes were cloned by the Gibson assembly method downstream the gene encoding the carrier into an expression cassette (SEQ N^{o} 82) present in the chloroplast transformation vector pAU76 linearized by PmeI to give respectively, pLA03 and pLA04.

### Transformation of Algae

The transformation vectors pAL01, pLA02, pLA03 and pLA04 were bombarded in C. reinhardtii cell (137c and CW15) as described in the Example 1.

In order to identify stable integration of the recombinant genes encoding fusion protein into the chloroplast algal genome, spectinomycin resistant colonies were screened by PCR analysis using the primers O5′ASTatpA2 (SEQ ID N^{o} 67) and O3′SUTRpsbD (SEQ ID N^{o} 54) annealing, respectively, in the atpA 3′UTR and psbD 5′UTR.

### Analyses and Results

Western Blot analysis performed using anti-Flag antibody on total soluble proteins extracted from different independent strains of LA01, LA03 or LA04 transformants revealed that the fusion proteins HA-SP-3F-FX-APRO-F-ELP4 (SEQ ID N^{o} 87), HA-SP-3F-FX-APRO-LGM-TV-ELPE4 (SEQ ID N^{o} 89) and HA-SP-3F-FX-APRO-LGM-EK-ELPE4 (SEQ ID N^{o} 91) were produced in the C. reinhardtii chloroplast.

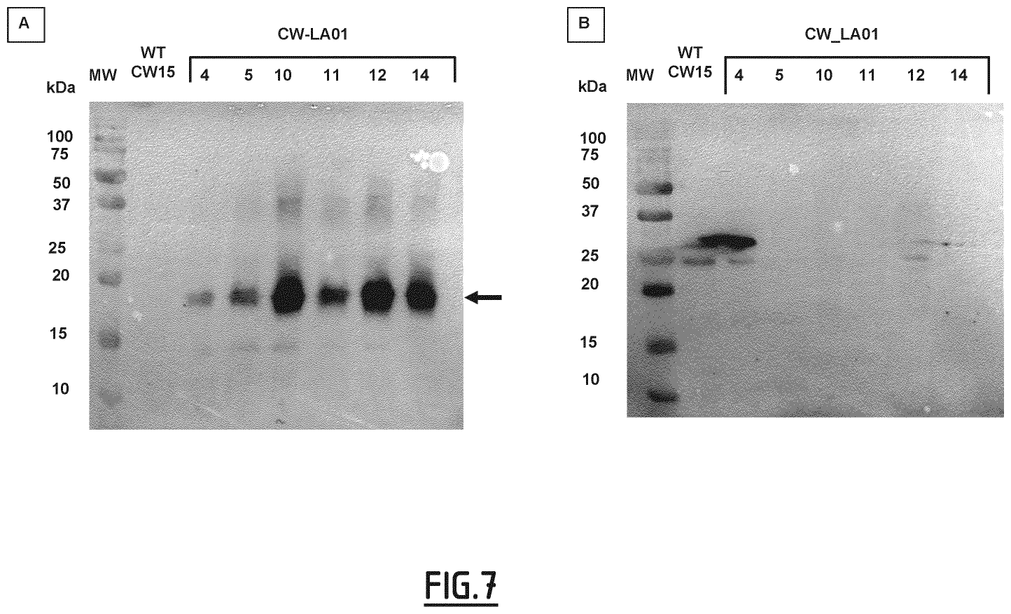

As shown in the FIG. 7, Western Blot analysis showed that the ELP4 polypeptide fused to 3F-FX-APRO is very well produced in the LA01 transformants using anti-Flag antibody. Moreover, in all LA01 transformants, the HA epitope Tag and the signal peptide seems to be cleaved because Western Blots performed on the same total soluble protein extracts showed that the primary anti-HA antibody didn't recognize any fusion protein (FIG. 7).

In the LA02 transformants, no recombinant protein were detected (FIG. 7) demonstrating that the fusion of ELP4 to 3F-FX-APRO (or aprotinin as a general manner) allows the accumulation of ELP4.

Biomass of one transformant CW-LA01 was produced. Cell pellet was resuspended in sonication buffer.

Fusion protein were purified by anti-Flag M2 affinity chromatography. Elution fraction containing the fusion protein were identify by Western Blot analysis, dialyzed and concentrated. Enterokinase protease digestions were performed followed by a size exclusion chromatography (HiLoad 26/00 Superdex 30) allowing the purification of the polypeptide ELP4.

The same method was applied for the purification of the ELPE4. The fusion protein were purified by affinity chromatography. Elution fraction containing the fusion protein were identify by Western Blot analysis, dialyzed and concentrated. Enterokinase or TEV protease digestions were performed depending on the transformant followed by a size exclusion chromatography (HiLoad 26/00 Superdex 30) allowing the purification of the polypeptide ELPE4.

In the case of LA03 transformants and after the TEV protease digestion, the released polypeptide was GVGVAPGEVGVAPGEVGVAPGEVGVAPGE (SEQ ID N^{o} 53).

In order to cleave by endoproteinase the polypeptides ELPE4 into peptides VGVAPGE, the SEC elution fractions were evaporated and dialyzed for salts removing and buffer changing, using a dialysis tube with a 1 kDa cutoff.

After digestion by the Glu-C_endoproteinase of the dialyzed samples as described in the Example 1, the released peptides were purified by a size exclusion chromatography.

1. A recombinant microalgae comprising a nucleic acid sequence encoding a recombinant protein, polypeptide or peptide comprising repeat units of amino acids, said protein, polypeptide or peptide being chosen from collagen, elastin and their derivatives, and said nucleic acid sequence being located in the chloroplast genome of microalgae.

2. A method for producing a recombinant protein, polypeptide or peptide comprising repeat units of amino acids in the chloroplast of microalgae, said protein, polypeptide or peptide being chosen from collagen, elastin and their derivatives, wherein said method comprises transformation of the chloroplast genome of microalgae with a nucleic acid sequence encoding said recombinant protein, polypeptide or peptide.

3. The method according to claim 2, comprising:
(i) providing a nucleic acid sequence encoding said recombinant protein, polypeptide or peptide;
(ii) introducing the nucleic acid sequence according to (i) into an expression vector which is capable of expressing the nucleic acid sequence; and
(iii) transforming the chloroplast genome of microalgae host cell by the expression vector.

4. The method according to claim 3, further comprising:
(iv) identifying the transformed microalgae host cell;
(v) characterizing the microalgae host cell for the production of recombinant protein, polypeptide or peptide expressed from said nucleic acid sequence;
(vi) extracting the recombinant protein, polypeptide or peptide; and optionally
(vii) purifying the recombinant protein, polypeptide or peptide.

5. The method according to claim 3, wherein said expression vector also comprises at least one expression cassette, said at least one expression cassette comprising the nucleic acid sequence encoding said recombinant protein, polypeptide or peptide.

6. The method according to claim 2, wherein the nucleic acid sequence encoding the protein, polypeptide or peptide is codon optimized for expression in the chloroplast genome of the microalgae host cell.

7. The method according to claim 1, wherein said protein, polypeptide or peptide derivative consists in an amino acid sequence at least 80% identical to the amino acid sequence of the recombinant peptide, polypeptide or protein.

8. The method according to claim 3, wherein said nucleic acid sequence encoding a recombinant protein, polypeptide or peptide is fused operationally at its 5′ or 3′end to a nucleic acid sequence encoding a carrier.

9. The method according to claim 3, wherein said nucleic acid sequence encoding a recombinant protein, polypeptide or peptide is operably linked to at least one regulatory sequence chosen from the psbD promoter and 5′UTR or the 16S rRNA promoter (Prrn) promoter fused with the atpA 5′UTR, the psaA promoter and 5′UTR, the atpA promoter and 5′ UTR and the atpA and rbcL 3′UTRs.

10. The method according to claim 5, wherein said at least one expression cassette further comprises a nucleic acid sequence encoding an epitope Tag peptide fused operationally at its 5′ or 3′end to the said nucleic acid sequence encoding the recombinant protein, polypeptide or peptide.

11. The method according to claim 5, wherein said at least one expression cassette further comprises a nucleic acid sequence encoding a signal peptide.

12. The method according to claim 5, wherein said at least one expression cassette further comprises a nucleic acid sequences encoding an amino acid sequence allowing the production of the said recombinant protein, polypeptide or peptide in specific cell compartment.

13. The method according to claim 2, wherein said microalgae is chosen from the group consisting of Chlorophyta, Chlorophyceae, Pleurastrophyceae, Prasinophyceae, Chromophyta, Bacillariophyceae (diatoms), Chrysophyceae, Phaeophyceae, Eustigmatophyceae, Haptophyceae, Raphidophyceae, Xanthophyceae, Cryptophyta, Cryptophyceae, Rhodophyta, Porphyridiophycea, Stramenopiles, Glaucophyta, Glaucocystophyceae, Chlorarachniophyceae, Haptophyceae, Dinophyceae, Scenedesmaceae, Euglenophyta, Euglenophyceae.

14. The method according to claim 13, wherein said microalgae is chosen from the group consisting of Chlamydomonas, Chlorella, Dunaliella, Haematococcus, diatoms, Scenedesmaceae, Tetraselmis, Ostreococcus, Porphyridium, and Nannochloropsis.

15. A method for producing a recombinant protein, polypeptide or peptide comprising repeat units of amino acids, said protein, polypeptide or peptide being chosen from collagen, elastin and their derivatives comprising the use of a recombinant microalgae according to claim 1.

16. The recombinant microalgae according to claim 1, wherein the nucleic acid sequence encoding the protein, polypeptide or peptide is codon optimized for expression in the chloroplast genome of the microalgae host cell.

17. The recombinant microalgae according to claim 1, wherein said protein, polypeptide or peptide derivative consists in an amino acid sequence at least 80% identical to the amino acid sequence of the recombinant peptide, polypeptide or protein.

18. The recombinant microalgae according to claim 1, wherein said nucleic acid sequence encoding a recombinant protein, polypeptide or peptide is fused operationally at its 5′ or 3′end to a nucleic acid sequence encoding a carrier.

19. The recombinant microalgae according to claim 1, wherein said nucleic acid sequence encoding a recombinant protein, polypeptide or peptide is operably linked to at least one regulatory sequence chosen from the psbD promoter and 5′UTR or the 16S rRNA promoter (Prrn) promoter fused with the atpA 5′UTR, the psaA promoter and 5′UTR, the atpA promoter and 5′ UTR and the atpA and rbcL 3′UTRs.

20. The recombinant microalgae according to claim 1, wherein said microalgae is chosen from the group consisting of Chlorophyta, Chlorophyceae, Pleurastrophyceae, Prasinophyceae, Chromophyta, Bacillariophyceae (diatoms), Chrysophyceae, Phaeophyceae, Eustigmatophyceae, Haptophyceae, Raphidophyceae, Xanthophyceae, Cryptophyta, Cryptophyceae, Rhodophyta, Porphyridiophycea, Stramenopiles, Glaucophyta, Glaucocystophyceae, Chlorarachniophyceae, Haptophyceae, Dinophyceae, Scenedesmaceae, Euglenophyta, Euglenophyceae.
